(19) United States
(12) Patent Application Publication
(10) Pub. No.: US 2023/0167181 A1
(43) Pub. Date: Jun. 1, 2023

(54) PHARMACEUTICAL COMPOSITIONS AND METHODS OF MANUFACTURE USING THERMALLY CONDUCTIVE EXCIPIENTS

(71) Applicant: BOARD OF REGENTS, THE UNIVERSITY OF TEXAS SYSTEM, Austin, TX (US)

(72) Inventors: Robert O. WILLIAMS, III, Austin, TX (US); Daniel A. DAVIS, Jr., Austin, TX (US)

(73) Assignee: BOARD OF REGENTS, THE UNIVERSITY OF TEXAS SYSTEM, Austin, TX (US)

(21) Appl. No.: 17/997,260

(22) PCT Filed: Apr. 27, 2021
(86) PCT No.: PCT/US2021/029283

§ 371 Date: Oct. 27, 2022

Related U.S. Application Data
(60) Provisional application No. 63/016,164, filed on Apr. 27, 2020.

(51) Int. Cl. C07K 16/28; A61K 47/69; A61K 47/02; A61K 47/32

(52) U.S. Cl. CPC C07K 16/2866; A61K 47/6939; A61K 47/02; A61K 47/32

(57) ABSTRACT

The present disclosure provides pharmaceutical compositions comprising a thermally conductive excipient which may be used to improve the heat transfer within the pharmaceutical compositions in a high energy mixing process. The resultant pharmaceutical compositions may be amorphous in nature and improve the processability of thermally labile or shear sensitive active agents.

This application claims the benefit of priority to U.S. Provisional Application No. 63/016,164, filed on Apr. 27, 2020, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a pharmaceutical composition with thermally conductive excipients.

### 2. Description of Related Art

An increasing number of drugs from contemporary pharmaceutical pipelines have poor solubility and low bioavailability, thus classified as Biopharmaceutics Classification System (BCS) II and IV (Ku and Dulin, 2012; Loftsson and Brewster, 2010) Over the past 10 years, the pharmaceutical industry has introduced amorphous solid dispersions (ASDs) as a viable formulation technique to address these challenges. Two methods, namely hot melt extrusion (HME) and spray drying (SD), are used for the majority of these drugs formulated as an ASD drugs (Jermain et al., 2018), but these processes have significant limitations. Innovative techniques [e.g., thin film freezing (TFF), KinetiSol Processing (KSD), and micro precipitated bulk powder (MBP)] have been developed to overcome the reported process limitations of HME and SD in order to further expand the process design space for poorly water soluble drugs (Zhang et al., 2012; Shah et al., 2013; Hughey et al., 2011).

KinetiSol processing (KinetiSol) is a high-energy, thermokinetic mixing process designed to produce amorphous solid dispersion compositions that overcome the processing and formulation limitations associated with SD and HME (Hughey et al., 2011; Miller et al., 2017; Miller et al., 2016a; Hughey et al., 2010; Miller et al., 2019). Increasing molecular weight and lipophilicity of drugs in development (Bergstrom et al., 2016; Leeson, 2016) severely limits their solubility in organic solvents, thus restricting SD as a formulation process (Jermain et al., 2018; Miller et al., 2016b). KinetiSol processing is not limited if the drug is poorly soluble in organic solvents, such as required by SD, thus further expanding the formulation space (Miller et al., 2017). HME has process limitations for formulating ASDs containing drugs having high melting points (e.g., >200° C.), are thermally labile or contain viscous polymers (Repka et al., 1999; LaFountaine et al., 2016a; Keen et al., 2013; Gupta et al., 2016a; Meena et al., 2016; Parikh et al., 2016; Gupta et al., 2016b). KinetiSol processing's ability to rapidly and efficiently mix minimizes the time the drug is exposed to elevated temperature (e.g., at temperatures greater than the T_{g} of the polymer carrier), thus minimizing or preventing chemical degradation caused from prolonged exposure of the drug to these high temperatures during HME (LaFountaine et al., 2016; Miller and Keen, 2014; Jermain et al., 2019). In contrast to HME, KinetiSol processing is not limited by torque and has exhibited successful formulation of viscous polymers that were previously thought unprocessable (Miller et al., 2017; LaFountaine et al., 2016a; Alexy et al., 2004; Brough et al., 2016a; Brough et al., 2016b; LaFountaine et al., 2016b; DiNunzio et al., 2010a). KinetiSol processing has demonstrated its ability to expand the formulation space for ASDs (Hughey et al., 2011; Miller et al., 2017; Miller et al., 2016a; Hughey et al., 2010; Miller et al., 2019; LaFountaine et al., 2016a; Keen et al., 2013; Brough et al., 2016a; Brough et al., 2016b; LaFountaine et al., 2016b; DiNunzio et al., 2010a). Currently, there are drugs that have no heat tolerance and have been difficult to formulate using KinetiSol processing (Ellenberger et al., 2018a).

To better understand this limitation for these drugs, it's important to delve more deeply into the KinetiSol process itself. Ellenberger et al (2018) published a review that describes the KinetiSol process in detail (Ellenberger et al., 2018a). Briefly, KinetiSol processing is a high-energy, fusion based approach that utilizes protruding blades to apply friction and shear to increase the composition's temperature. The energy input into the system and speed of the blades control how rapidly the composition within the chamber increases in temperature. The energy input is transferred from the blades to the composition, thus as a consequence of the composition's inability to dissipate this energy, a rapid rise in the composition's temperature is typically observed (LaFountaine et al., 2016b; Ellenberger et al., 2018a). For most drugs contained in compositions that are processed by KinetiSol processing, significant benefits from this very rapid increase in temperature as a result of high shear mixing reduces the incidence for drug degradation (Keen et al., 2013; Miller and Keen, 2014).

The impact of the different Kinetisol processing parameters (e.g., blade speed (RPM), temperature and time) on the processing profile of a composition has been reported. LaFountaine et al., (2016) observed the relationship between a composition's temperature and processing time as a function of blade RPM, as shown in FIG. 1. Minimizing exposure to elevated temperatures by prolonging the mixing time at a lower temperature and lower blade RPM is advantageous for drugs that are sensitive to high shear and/or are thermally labile (Miller and Keen, 2014). Even by manipulating KinetiSol processing parameters, not all compositions have been able to achieve prolonged mixing at a selected constant temperature.

Polymers have intrinsically have poor conductive properties (Yang, 2007) such that when exposed to kinetic energy increases, the composition's temperature can be explained by the first law of thermodynamics the energy input into the composition is greater than the energy output of the composition resulting in the composition increasing in temperature. This increase in energy causes the composition to heat to higher temperatures leading to degradation of the pharmaceutical composition. Therefore, there remains a need to develop new pharmaceutical compositions and methods to prepare them which reduce the destruction of thermally and shear sensitive compositions during processing.

### SUMMARY OF THE INVENTION

The present disclosure provides pharmaceutical compositions which comprise a thermally conductive excipient. Without wishing to be bound by any theory, the present pharmaceutical compositions may result in compositions which are more stable against degradation of the active agent. The active agent may be one that is poorly soluble or may be one that undergoes chemical degradation after being exposed to heat or shear stress.

In some aspects, the present disclosure provides methods of preparing a pharmaceutical composition comprising subjecting:
(A) an active agent;
(B) a pharmaceutically acceptable polymer; and
(C) a thermally conductive excipient;
   to a high energy mixing process at a first temperature and a set speed to obtain the pharmaceutical composition. In some embodiments, the pharmaceutical composition comprises an amorphous active agent. In some embodiments, the pharmaceutical composition comprises an amorphous solid dispersion. In some embodiments, the active agent is a poorly soluble drug. In some embodiments, the active agent is a BCS class 2 drug. In some embodiments, the active agent is a BCS class 3 drug. In some embodiments, the active agent is a BCS class 4 drug. In some embodiments, the active agent is an agent which undergoes degradation at an elevated temperature in a formulation process. In some embodiments, the active agent is chemically sensitive to temperature. In some embodiments, the active agent is chemically sensitive to shear. In some embodiments, the active agent does not undergo degradation in the high energy mixing process when the thermally conductive excipient is added to the pharmaceutical composition. In some embodiments, the active agent is an agent with a melting point of greater than 200° C.

In some embodiments, the active agent is selected from anticancer agents, antifungal agents, psychiatric agents such as analgesics, consciousness level-altering agents such as anesthetic agents or hypnotics, nonsteroidal anti-inflammatory agents (NSAIDS), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives. In some embodiments, the active agent is an anti-inflammatory agent. In some embodiments, the anti-inflammatory agent is a CCR1 antagonist. In other embodiments, the active agent is an antiepileptic such as sodium channel blocker. In some embodiments, the pharmaceutical composition comprises from about 5% w/w to about 90% w/w of the active agent. In some embodiments, the pharmaceutical composition comprises from about 10% w/w to about 50% w/w of the active agent. In some embodiments, the pharmaceutical composition comprises from about 20% w/w to about 40% w/w of the active agent. In some embodiments, the pharmaceutical composition comprises from about 50% w/w to about 90% w/w of the active agent. In some embodiments, the pharmaceutical composition comprises from about 60% w/w to about 80% w/w of the active agent. In some embodiments, the pharmaceutical composition is substantially, essentially, or entirely free of any other active agent.

In some embodiments, the pharmaceutically acceptable polymer is a cellulosic polymer. In some embodiments, the cellulosic polymer is a neutral cellulosic polymer. In other embodiments, the cellulosic polymer is a charged cellulosic polymer. In some embodiments, the cellulosic polymer is hypromellose acetate succinate. In some embodiments, the pharmaceutically acceptable polymer is a neutral non-cellulosic polymer. In further embodiments, the neutral non-cellulosic polymer comprises a poly(vinyl acetate), polyvinyl caprolactam, poly(vinylpyrrolidone), poly(ethylene glycol), poly(ethylene oxide), poly(vinyl alcohol), or methacrylate unit. In some embodiments, the pharmaceutically acceptable polymer comprises a poly(vinyl acetate) or a methacrylate unit. In some embodiments, the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate. In some embodiments, the pharmaceutical composition comprises from about 5% w/w to about 90% w/w of the pharmaceutically acceptable polymer. In some embodiments, the pharmaceutical composition comprises from about 30% w/w to about 80% w/w of the pharmaceutically acceptable polymer. In some embodiments, the pharmaceutical composition comprises from about 50% w/w to about 70% w/w of the pharmaceutically acceptable polymer. In some embodiments, the pharmaceutical composition is substantially, essentially, or entirely free of any other pharmaceutically acceptable polymer.

In some embodiments, the thermally conductive excipient is a material that leads to improved thermal conductivity. In some embodiments, the thermally conductive excipient is a material with a thermal conductivity of greater than 10 W/mK. In further embodiments, the thermal conductivity is greater than 100 W/mK. In still further embodiments, the thermal conductivity is greater than 200 W/mK. In some embodiments, the thermal conductivity is from about 100 to about 400 W/mK. In some embodiments, the thermally conductive excipient is an inorganic material. In some embodiments, the thermally conductive excipient is an aluminum material. In further embodiments, the aluminum material is an aluminum inorganic salt. In still further embodiments, the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide. In yet further embodiments, the aluminum inorganic salt is potassium aluminum silicate with a coating of titanium dioxide and/or iron oxide. In some embodiments, the inorganic material is iron oxide, titanium oxide, or silicates. In some embodiments, the thermally conductive excipient is an organic material. In further embodiments, the organic material is a dye. In further embodiments, the dye is carmine, a phthalocyanine, or a diazo compound. In some embodiments, the pharmaceutical composition comprises from about 0.01% w/w to about 80% w/w of the thermally conductive excipient. In some embodiments, the pharmaceutical composition comprises from about 0.1% w/w to about 50% w/w of the thermally conductive excipient. In some embodiments, the pharmaceutical composition comprises from about 1% w/w to about 30% w/w of the thermally conductive excipient. In some embodiments, the pharmaceutical composition is substantially, essentially, or entirely free of any other thermally conductive excipient.

In some embodiments, the high energy mixing process does not comprise an external heat input. In some embodiments, the high energy mixing process is a KinetiSol process. In some embodiments, the set speed is from about 500 rpm to about 6000 rpm. In some embodiments, the set speed is from about 1000 rpm to about 5000 rpm. In some embodiments, the set speed is from about 2000 rpm to about 4000 rpm. In some embodiments, the high energy mixing process comprises mixing the composition comprising two or more set speeds. In some embodiments, the high energy mixing process is run for a set amount of time. In further embodiments, the set amount of time is less than 300 seconds. In still further embodiments, the set amount of time is from about 5 seconds to about 300 seconds. In yet further embodiments, the set amount of time is from about 5 seconds to about 60 seconds. In some embodiments, the high energy mixing process comprises a period of prolonged mixing wherein a second temperature does not change by more than 15° C. In further embodiments, the second temperature does not change by more than 10° C. In still further embodiments, the second temperature does not change by more than 5° C. In yet further embodiments, the second temperature does not change by more than 1° C. In some embodiments, the high energy mixing process comprises a glass transition inflection region and the region of prolonged mixing. In further embodiments, the glass transition inflection region and the region of prolonged mixing has a time period between the glass transition inflection region and the region of prolonged mixing of less than 20 seconds. In still further embodiments, the time period is less than 15 seconds. In yet further embodiments, the time period is less than 10 seconds. In some embodiments, the high energy mixing process is run until the composition reaches a specific elevated temperature. In further embodiments, the specific temperature is reached quicker than a composition without the thermally conductive excipient. In still further embodiments, the temperature is reached 30% faster. In yet further embodiments, the temperature is reach 10% faster.

In some embodiments, the method further comprises an excipient. In some embodiments, the pharmaceutical composition is substantially, essentially, or entirely free of any other excipient. In some embodiments, the pharmaceutical composition is substantially, essentially, or entirely free of any other compound. In some embodiments, the methods further comprise quenching the pharmaceutical composition. In some embodiments, the methods further comprise milling the pharmaceutical composition. In some embodiments, the methods further comprise formulating the pharmaceutical composition into a unit dose. In further embodiments, the unit dose is formulated for oral, pulmonary, nasal, topical, transdermal, or parenteral delivery. In still further embodiments, the unit dose is formulated for oral delivery. In yet further embodiments, the oral delivery is formulated as a tablet, capsule, or suspension. In some embodiments, the unit dose is formulated for topical delivery. In further embodiments, the topical delivery is an emulsion, ointment, or cream. In some embodiments, the unit dose is formulated for parenteral delivery. In further embodiments, the parenteral delivery is a suspension, microemulsion, or depot.

In other aspects, the present disclosure provides pharmaceutical compositions comprising:
(A) an active agent;
(B) a pharmaceutically acceptable polymer; and
(C) a thermally conductive excipient;
   wherein the pharmaceutical composition comprises an amorphous active agent and the pharmaceutical composition comprises a homogenous mixture of the active agent, the pharmaceutically acceptable polymer, and the thermally conductive excipient. In some embodiments, the pharmaceutical composition comprises an amorphous solid dispersion. In some embodiments, the active agent is a poorly soluble drug. In some embodiments, the active agent is a BCS class 2 drug. In some embodiments, the active agent is a BCS class 3 drug. In some embodiments, the active agent is a BCS class 4 drug. In some embodiments, the active agent is an agent which undergoes degradation at an elevated temperature in a formulation process. In some embodiments, the active agent is chemically sensitive to temperature. In some embodiments, the active agent is chemically sensitive to shear. In some embodiments, the active agent does not undergo degradation in the high energy mixing process when the thermally conductive excipient is added to the pharmaceutical composition. In some embodiments, the active agent is an agent with a melting point of greater than 200° C.

In some embodiments, the active agent is selected from anticancer agents, antifungal agents, psychiatric agents such as analgesics, consciousness level-altering agents such as anesthetic agents or hypnotics, nonsteroidal anti-inflammatory agents (NSAIDS), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives. In some embodiments, the active agent is an anti-inflammatory agent. In some embodiments, the anti-inflammatory agent is a CCR1 antagonist. In other embodiments, the active agent is an antiepileptic such as sodium channel blocker. In some embodiments, the pharmaceutical composition comprises from about 5% w/w to about 90% w/w of the active agent. In some embodiments, the pharmaceutical composition comprises from about 10% w/w to about 50% w/w of the active agent. In some embodiments, the pharmaceutical composition comprises from about 20% w/w to about 40% w/w of the active agent. In some embodiments, the pharmaceutical composition comprises from about 50% w/w to about 90% w/w of the active agent. In some embodiments, the pharmaceutical composition comprises from about 60% w/w to about 80% w/w of the active agent. In some embodiments, the pharmaceutical composition is substantially, essentially, or entirely free of any other active agent.

In some embodiments, the pharmaceutically acceptable polymer is a cellulosic polymer. In some embodiments, the cellulosic polymer is a neutral cellulosic polymer. In other embodiments, the cellulosic polymer is a charged cellulosic polymer. In some embodiments, the cellulosic polymer is hypromellose acetate succinate. In some embodiments, the pharmaceutically acceptable polymer is a neutral non-cellulosic polymer. In further embodiments, the neutral non-cellulosic polymer comprises a poly(vinyl acetate), polyvinyl caprolactam, poly(vinylpyrrolidone), poly(ethylene glycol), poly(ethylene oxide), poly(vinyl alcohol), or methacrylate unit. In some embodiments, the pharmaceutically acceptable polymer comprises a poly(vinyl acetate) or a methacrylate unit. In some embodiments, the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate. In some embodiments, the pharmaceutical composition comprises from about 5% w/w to about 90% w/w of the pharmaceutically acceptable polymer. In some embodiments, the pharmaceutical composition comprises from about 30% w/w to about 80% w/w of the pharmaceutically acceptable polymer. In some embodiments, the pharmaceutical composition comprises from about 50% w/w to about 70% w/w of the pharmaceutically acceptable polymer. In some embodiments, the pharmaceutical composition is substantially, essentially, or entirely free of any other pharmaceutically acceptable polymer.

In some embodiments, the thermally conductive excipient is a material that leads to improved thermal conductivity. In some embodiments, the thermally conductive excipient is a material with a thermal conductivity of greater than 10 W/mK. In further embodiments, the thermal conductivity is greater than 100 W/mK. In still further embodiments, the thermal conductivity is greater than 200 W/mK. In some embodiments, the thermal conductivity is from about 100 to about 400 W/mK. In some embodiments, the thermally conductive excipient is an inorganic material. In some embodiments, the thermally conductive excipient is an aluminum material. In further embodiments, the aluminum material is an aluminum inorganic salt. In still further embodiments, the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide. In yet further embodiments, the aluminum inorganic salt is potassium aluminum silicate with a coating of titanium dioxide and/or iron oxide. In some embodiments, the inorganic material is iron oxide, titanium oxide, or silicates. In some embodiments, the thermally conductive excipient is an organic material. In further embodiments, the organic material is a dye. In further embodiments, the dye is carmine, a phthalocyanine, or a diazo compound. In some embodiments, the pharmaceutical composition comprises from about 0.01% w/w to about 80% w/w of the thermally conductive excipient. In some embodiments, the pharmaceutical composition comprises from about 0.1% w/w to about 50% w/w of the thermally conductive excipient. In some embodiments, the pharmaceutical composition comprises from about 1% w/w to about 30% w/w of the thermally conductive excipient. In some embodiments, the pharmaceutical composition is substantially, essentially, or entirely free of any other thermally conductive excipient.

In some embodiments, the pharmaceutical composition has been processed through a high energy mixing process. In some embodiments, the composition further comprises an excipient. In some embodiments, the pharmaceutical composition is substantially, essentially, or entirely free of any other excipient. In some embodiments, the pharmaceutical composition is substantially, essentially, or entirely free of any other compound. In some embodiments, the composition is milled. In some embodiments, the composition is formulated into a unit dose. In further embodiments, the unit dose is formulated for oral, pulmonary, nasal, topical, transdermal, or parenteral delivery. In some embodiments, the unit dose is formulated for oral delivery. In further embodiments, the oral delivery is formulated as a tablet, capsule, or suspension. In some embodiments, the unit dose is formulated for topical delivery. In further embodiments, the topical delivery is an emulsion, ointment, or cream. In some embodiments, the unit dose is formulated for parenteral delivery. In further embodiments, the parenteral delivery is a suspension, microemulsion, or depot. In some embodiments, the composition comprises:
(A) about 3% w/w of the thermally conductive excipient, wherein the thermally conductive excipient is potassium aluminum silicate with a coating of titanium dioxide and/or iron oxide;
(B) about 33% w/w of the active agent, wherein the active agent is CCR1 antagonist, and
(C) about 64% w/w of the pharmaceutically acceptable polymer, wherein the pharmaceutically acceptable polymer is hypromellose acetate succinate.

In still other aspects, the present disclosure provides pharmaceutical compositions prepared according to the methods of the present disclosure.

In yet other aspects, the present disclosure provides methods of treating a disease or disorder comprising administering to the patient a therapeutically effective amount of a pharmaceutical composition of the present disclosure wherein the active agent is effective to treat the disease or disorder.

In other aspects, the present disclosure provides pharmaceutical compositions comprising:
(A) a pharmaceutically acceptable polymer; and
(B) a thermally conductive excipient;
   wherein the pharmaceutical composition comprises a homogenous mixture of the pharmaceutically acceptable polymer and the thermally conductive excipient.

In still other aspects, the present disclosure provides methods of preparing a composition comprising:
(A) a pharmaceutically acceptable polymer; and
(B) a thermally conductive excipient;
   to a high energy mixing process at a first temperature and a set speed to obtain the composition.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows DOE on how the control parameters' influence on the duration of the formulation process, initiation of baseline temperature increase and rate of temperature increase (with no TCE present in composition). See (LaFountaine et al., 2016b).

FIG. 2 shows the FTIR spectrum of the composition mixture showing that there is no change in the molecular interactions upon the addition of candurin to the pharmaceutical composition.

FIG. 3 shows HSPLM of various mixtures to determine the presence of crystallinity in the pharmaceutical compositions.

FIG. 4 shows change in temperature as a function of time for the processing of the composition at 3400 rpm.

FIG. 5 shows change in temperature as a function of time for the processing of the composition at 3400 rpm for a pharmaceutical composition with and without a thermally conductive excipient.

FIG. 6 shows change in temperature as a function of time for the processing of the composition at 2700 rpm for a pharmaceutical composition with and without a thermally conductive excipient.

FIG. 7 shows a powder x-ray diffraction spectrum of various pharmaceutical compositions which show that after processing through a high energy mixture the resultant pharmaceutical composition is an amorphous mixture as opposed to physical mixtures which retained crystallinity.

FIG. 8 shows the powder x-ray diffraction of various compositions including individual components and the physical mixture.

FIG. 9 shows the powder x-ray diffraction of several compositions over a narrow window of 5-35 degrees 2θ.

FIG. 10 shows the dissolution of the active agent in two different compositions at different pH as a function of time.

FIG. 11 shows scans from differential scanning calorimetry of the compositions made as reference samples from U.S. Patent Application Publication No. 2019/037441. The reference samples indicate the presence of crystallinity.

FIG. 12 shows change in temperature as a function of time for the processing of the composition at 3400 rpm.

FIG. 13 shows change in temperature as a function of time for the processing of the composition at 3400 rpm.

### DESCRIPTION OF ILLUSTRATIVE EMBODIMENTS

In some aspects of the present disclosure, the pharmaceutical compositions provided herein comprise a thermally conductive excipient that allows the composition to be processed through high energy processing in a manner wherein the heat is captured and slowly released into the composition thus allowing for complete mixing while maintaining lower temperature of the composition. Without wishing to be bound by any theory, the presently disclosed compositions may lead to reduced degradation of thermally or shear unstable compounds or improved solubility of compounds which are otherwise poorly soluble. Furthermore, it is contemplated that the pharmaceutical compositions may be easier to control the temperature where the thermokinetic mixing occurs or allows the temperature of the pharmaceutical composition to be increased at a faster rate and then held relatively constant. Methods of preparing these compositions are described in more detail therein.

### I. PHARMACEUTICAL COMPOSITIONS

In some aspects, the present disclosure provides pharmaceutical compositions containing an active agent such as an active pharmaceutical ingredient or a pharmaceutically acceptable salt, ester, derivative, analog, pro-drug, or solvates thereof, a pharmaceutically acceptable polymer including polymeric excipients, and a thermally conductive excipient (TCE) such as an inorganic or organic compound which promotes heat transfer. These compositions may be amorphous in nature and formulated as an amorphous solid dispersion. In some aspects, the pharmaceutically acceptable polymer and the thermally conductive excipient may be processed including through a high energy process to obtain a compound excipient which is then formulated with the active agent. In some embodiments, the pharmaceutical composition is substantially, essentially, or entirely free of any other compound.

In some aspects, the present composition may be substantially, essentially, or entirely free from any plasticizer or similar agents which interact with the pharmaceutical composition on the molecular level. Without wishing to be bound by any theory, it is believed that the thermally conductive excipient does not interact with the pharmaceutical composition but rather acts to facilitate the transfer of heat more efficiently.

A. Active Agent

The pharmaceutical compositions described herein comprise an active agent. The pharmaceutical compositions described herein contain an active agent in an amount between about 10% to about 90% w/w, between about 20% to about 80% w/w, between about 20% to about 70% w/w, or between about 25% to about 50% w/w of the total composition. In some embodiments, the amount of the active agent is from about 10%, 20%, 22%, 24%, 25%, 26%, 28%, 30%, 32%, 34%, 35%, 36%, 38%, 40%, 42%, 44%, 45%, 50%, 55%, 60%, 65%, 70%, 75%, 80%, to about 90% w/w or any range derivable therein. In some embodiments, the pharmaceutical composition is substantially, essentially, or entirely free of any other active agent.

In some embodiments, the active agent is classified using the Biopharmaceutical Classification System (BCS), originally developed by G. Amidon, which separates pharmaceuticals for oral administration into four classes depending on their aqueous solubility and their permeability through the intestinal cell layer. According to the BCS, drug substances are classified as follows: Class I—High Permeability, High Solubility; Class II—High Permeability, Low Solubility; Class III—Low Permeability, High Solubility; and Class IV—Low Permeability, Low Solubility.

In particular, typical BCS Class II that may be incorporated into the present pharmaceutical compositions include but are not limited to anti-infectious drugs such as Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconalzole, Lopinavir, Mebendazole, Nelfinavir, Nevirapine, Niclosamide, Praziquantel, Pyrantel, Pyrimethamine, Quinine, and Ritonavir. Antineoplasic drugs such as Bicalutamide, Cyproterone, Gefitinib, Imatinib, and Tamoxifen. Biologic and Immunologic Agents such as Cyclosporine, Mycophenolate mofetil, Tacrolimus. Cardiovascular Agents such as Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nilvadipine, Nisoldipine, Simvastatin, Spironolactone, Telmisartan, Ticlopidine, Valsartan, Verapamil, Warfarin. Central Nervous System Agents such as Acetaminophen, Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam, Meloxicam, Metaxalone, Methylphenidate, Metoclopramide, Nicergoline, Naproxen, Olanzapine, Oxcarbazepine, Phenytoin, Quetiapine Risperidone, Rofecoxib, and Valproic acid. Dermatological Agents such as Isotretinoin—Endocrine and Metabolic Agents such as Dexamethasone, Danazol, Epalrestat, Gliclazide, Glimepirjde, Glipizide, Glyburide (glibenclamide), levothyroxine sodium, Medroxyprogesterone, Pioglitazone, and Raloxifene. Gastrointestinal Agents such as Mosapride, Orlistat, Cisapride, Rebamipide, Sulfasalazine, Teprenone, and Ursodeoxycholic Acid. Respiratory Agents such as Ebastine, Hydroxyzine, Loratadine, and Pranlukast. However, the skilled person will be well aware of other BCS class II drugs which can be used with the pharmaceutical compositions described herein.

Additionally, BCS class III drugs that may be incorporated into the present pharmaceutical compositions include but are not limited to cimetidine, acyclovir, atenolol, ranitidine, abacavir, captopril, chloramphenicol, codeine, colchicine, dapsone, ergotamine, kanamycin, tobramycin, tigecycline, zanamivir, hydralazine, hydrochlorothiazide, levothyroxine, methyldopa, paracetamol, propylthiouracil, pyrodostigmine, sodium cloxacillin, thiamine, benzidazole, didanosine, ethambutol, ethosuximide, folic acid, nicotinamide, nifurtimox, and salbutamol sulfate. However, the skilled person will be well aware of other BCS class III drugs which can be used with the pharmaceutical compositions described herein.

Additionally, BCS class IV drugs that may be incorporated into the present pharmaceutical compositions include but are not limited to hydrochlorothiazide, furosemide, cyclosporin A, itraconazole, indinavir, nelfinavir, ritonavir, saquinavir, nitrofurantoin, albendazole, acetazolamide, azithromycin, senna, azathioprine, chlorthalidone, BI-639667, rifabutin, paclitaxel, curcumin, etoposide, neomycin, methotrexate, atazanavir sulfate, Aprepitant, amphotericin B, amiodarone hydrochloride, or mesalamine. However, the skilled person will be well aware of other BCS class IV drugs which can be used with the pharmaceutical compositions described herein.

While the pharmaceutical compositions and methods described herein can be applied to any BCS class of drugs, BCS class II and IV are of interest for the pharmaceutical compositions described herein. Additionally, other active agents that are of specific consideration are those are those that are high melting point drugs such as a drug that has a melting point of greater than 200° C. Alternatively, the active agents used herein may have a melting point from about 25° C. to about 1,000° C., from about 100° C. to about 750° C., or from about 200° C. to about 500° C. In particular, the melting point may be greater than 200° C., 250° C., 300° C., 400° C., 500° C., 300° C., 700° C., 750° C., 800° C., 900° C., or 1,000° C.

In some aspects, the present methods may be used to formulate one or more poorly soluble active agents such as deferasirox, etravirine, indomethacin, posaconazole, and ritonavir. Etravirine is a neutral active agent and may be used as a model for other neutral active agents. Deferasirox and indomethacin is a weak acid API and may be used as a model for other weak acid APIs. Posaconazole, itraconazole, and ritonavir are weak base APIs and may be used as models for other weak base APIs.

Suitable active agents may be any poorly water-soluble, biologically active agents or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal antiinflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the active agents may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, niclosamide, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the active agents may be busulfan, taxane or other anticancer agents; or alternatively, itraconazole (Itra) and posaconazole (Posa) or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, Posaconazole, voriconazole, terconazole and c) thiazoles such as abafungin. Other active agents that may be used with this approach include, but are not limited to, hyperthyroid drugs such as carimazole, anticancer agents like cytotoxic agents such as epipodophyllotoxin derivatives, taxanes, bleomycin, anthracyclines, as well as platinum compounds and camptothecin analogs. The following active agents may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The active agents may also include a psychiatric agent such as an antipsychotic, anti-depressive agent, or analgesic and/or tranquilizing agents such as benzodiazepines. The active agents may also include a consciousness level-altering agent or an anesthetic agent, such as propofol. The present compositions and the methods of making them may be used to prepare a pharmaceutical composition with the appropriate pharmacokinetic properties for use as therapeutics.

In some aspects, the method may be mostly used with active agents which undergo degradation at an elevated temperature or pressure/shear. The active agents that may be used include those which decompose at a temperature above about 50° C. In some embodiments, the active agent decomposes above a temperature of 80° C. In some embodiments, the active agent decomposes above a temperature of 100° C. In some embodiments, the active agent decomposes above a temperature of 150° C. The active agent that may be used include therein which decompose at a temperature of greater than about 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., or 150° C.

Alternatively, the compound may be one that is sensitive to shear. These compounds are compounds for which the chemical and/or physical properties may change due to friction resulting from the manufacturing process itself, including chemical degradation of a drug or the loss of molecular weight of a polymer as non-limiting examples. The degree of loss of the chemical or physical properties of a compound due to shear is often seen as a function of the degree of mixing (e.g., blade RPM, rotation speed) and the properties of the polymer carrier (e.g. rheological properties).

B. Excipients

In some aspects, the present disclosure comprises one or more excipients formulated into pharmaceutical compositions including a pharmaceutically acceptable polymer and a thermally conductive excipient. An “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Non-limiting examples of excipients include polymer carriers, stabilizing agents, surfactants, surface modifiers, solubility enhancers, buffers, encapsulating agents, antioxidants, preservatives, nonionic wetting or clarifying agents, viscosity increasing agents, and absorption-enhancing agents. In some embodiments, the pharmaceutical composition is substantially, essentially, or entirely free of any other excipient.

1. Thermally Conductive Excipients

In some aspects, the pharmaceutical composition may further comprise one or more inorganic or organic material that promotes heat transfer. These materials may be described as a “thermally conductive excipient” or a “TCE.” In one embodiment, the thermally conductive excipient is inert and does not interact with the formulation. Without wishing to be bound by any theory, it is believed that the addition of the thermally conductive excipient increases the ability of the system to readily disperse energy throughout the formulation. By increasing the efficiency of heat transfer within a thermokinetic or high energy mixing process, it is believed that the addition eliminates chemical degradation by decreasing: the maximum required temperature, the overall exposure to elevated temperatures, and/or the amount of shear applied to the system. The addition of these materials thus may be used to create a more favorable formation of an amorphous material.

In some embodiments, the pharmaceutical compositions of the present disclosure include one or more inorganic and/or organic materials as the thermally conductive excipient. Some non-limiting examples of TCEs include: Candurin® (potassium aluminum silicate (mica) with a coating of Titanium dioxide and/or iron oxide), Potassium aluminum silicate (PAS), aluminum, aluminum sulfates, sodium aluminum phosphate acidic, sodium aluminum silicate, calcium aluminum silicate, bentonite, starch aluminum octenyl succinate and other aluminum consisting composition. A skilled artisan would be aware of such aluminum based TCEs which may be used in the pharmaceutical compositions described herein. The pharmaceutically acceptable polymers used herein generally have low thermal conductivity of around 0.1-0.6 W/(m K) while aluminum based excipients and similar TCEs may be 2 magnitudes higher ˜200 W/mK. See Yang et al. In some embodiments, the TCE may have a thermal conductivity of greater than about 25 W/mK, 50 W/mK, 100 W/mK, 125 W/mK, 150 W/mK, 160 W/mK, 170 W/mK, 180 W/mK, 190 W/mK, or greater than about 200 W/mK. Some other non-limiting examples of inorganic thermally conductive excipients that may be used include iron oxide, titanium oxide, silicates. In other embodiments, the TCE may be an organic material, such as a dye. Some non-limiting examples of dyes which may be used include carmine, phtlocyanine and diazos. Additional TCEs may be obtained from those used in the semiconductor field but such excipients must be considered with some caution because those excipients may not be amenable to introduction into a pharmaceutical composition for human delivery or may lead to degradation of the active agent. Such TCEs are described in U.S. Pat. Nos. 5,900,447, 9,434,870, and U.S. Patent Application No. 2006/0084742

In some embodiments, the TCE is a compound or composition that is already an FDA approved excipient for human consumption. One example of a TCE that is approved for human consumption and may be incorporated within the pharmaceutical composition is Candurin®. Candurin® is not soluble in water or other biorelevant conditions making it not be completely digested upon consumption but rather only subject to extraction by stomach acids. Candurin® and other aluminum derivatives are often used as commercially available food additive in confections, candy, decorations and beverages at maximum concentrations of 1.25%, equating to a range of 10 mg/kg-323 mg/kg/day. Candurin® contains pearlescent pigments achieve there different coloring effects by using different degrees of titanium oxide and/or iron oxide around a potassium aluminum silicate (PAS) core. The pearlescent color effect results from the partial transmittance and partial reflection of light as well as interference of light through the platelets. PAS-BPP comes in three types all types (types I-III) and may be used in this application. In particular, it is noted that PAS-BPP are expected to have excellent thermal stability during food processing and storage, as the thermal conditions experienced are mild in comparison to which the PAS-BPP is made (900 degree Celsius). Therefore, any Candurin® may be used in this application.

Furthermore, the pharmaceutical composition described herein have a concentration of the thermally conductive excipient ranging from about 0.01% to about 80% w/w. In some embodiments, the amount of thermally conductive excipient is from about 0.1% to about 80% w/w, from about 0.5% to about 50% w/w, 1% to about 40% w/w, or 2% to about 10% w/w. The amount of thermally conductive excipient may be from about 1%, 2%, 3%, 4%, 5%, 6%, 7%, 8%, 9%, 10%, 15%, 20%, 25%, 30%, 40%, 50%, 60%, 70%, to about 80%, or any range derivable therein. In some embodiments, the pharmaceutical composition is substantially, essentially, or entirely free of any other thermally conductive excipient.

2. Pharmaceutically Acceptable Polymers

In some aspects, the present disclosure provides compositions which may further comprise a pharmaceutically acceptable polymer. In some embodiments, the polymer (polymer carrier) has been approved for use in a pharmaceutical formulation and is known to undergo softening or increased pliability when raised above a specific temperature without substantially degrading.

When a pharmaceutically acceptable polymer is present in the composition, the pharmaceutically acceptable polymer is present in the composition at a level between 1% to 90% w/w, between 10% to 80% w/w, between 20% to 70% w/w, between 30% to 70% w/w, between 40% to 60% w/w. In some embodiments, the amount of the pharmaceutically acceptable polymer is from about 5%, 10%, 15%, 50%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, to about 90% w/w or any range derivable therein. In some embodiments, the pharmaceutical composition is substantially, essentially, or entirely free of any other pharmaceutically acceptable polymer.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups which are charged at a physiologically relevant pH. Some non-limiting examples of neutral non-cellulosic polymers include polyvinyl pyrrolidone, polyvinyl alcohol, copovidone, and poloxamer Within this class, in some embodiments, pyrrolidone containing polymers are particularly useful. Some non-limiting examples of charged cellulosic polymers include cellulose acetate phthalate and hydroxypropyl methyl cellulose acetate succinate. Finally, some non-limiting examples of neutral cellulosic polymers include hydroxypropyl cellulose, hydroxypropyl methyl cellulose, hydroxyethyl cellulose, and hydroxymethyl cellulose.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate—methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate—methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer such as SoluPlus® (PEG 6000/vinylcaprolactam/vinyl acetate (13:57:30)), polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

Additional pharmaceutically acceptable polymers that may be used in the presently disclosed pharmaceutical compositions include but are not limited to polyethylene oxide; polypropylene oxide; polyvinylpyrrolidone; polyvinylpyrrolidone-co-vinylacetate; acrylate and methacrylate copolymers; polyethylene; polycaprolactone; polyethylene-co-polypropylene; alkylcelluloses such as methylcellulose; hydroxyalkylcelluloses such as hydroxymethylcellulose, hydroxyethylcellulose, hydroxypropylcellulose, and hydroxybutylcellulose; hydroxyalkyl alkylcelluloses such as hydroxyethyl methylcellulose and hydroxypropyl methylcellulose; starches, pectins; polysaccharides such as tragacanth, gum arabic, guar gum, and xanthan gum. One embodiment of the pharmaceutically acceptable polymer is poly(ethylene oxide) (PEO), which can be purchased commercially from companies such as the Dow Chemical Company, which markets PEO under the POLY OX® exemplary grades of which can include WSR N80 having an average molecular weight of about 200,000; 1,000,000; and 2,000,000.

3. Other Excipients

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active agent in a given solution, so that the particles formed contain more than one active agent.

Any pharmaceutically acceptable excipient known to those of skill in the art may be used to produce the pharmaceutical compositions disclosed herein. Examples of excipients for use with the present disclosure include, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used in the pharmaceutical composition to enhance the efficacy and efficiency of the active agent in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers and absorption enhancers. The excipients may be chosen to modify the intended function of the active ingredient by improving flow, or bioavailability, or to control or delay the release of the API. Specific nonlimiting examples include: sucrose, trehaolose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphadityl choline, glycolic acid and salts, deoxycholic acid and salts, sodium fusidate, cyclodextrins, polyethylene glycols, Labrasol®, polyvinyl alcohols, polyvinyl pyrrolidones and tyloxapol.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or combination thereof, and plasticizers including citrate esters, polyethylene glycols, PG, triacetin, diethylphthalate, castor oil, and others known to those or ordinary skill in the art. Extruded material may also include an acidifying agent, adsorbent, alkalizing agent, buffering agent, colorant, flavorant, sweetening agent, diluent, opaquant, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer carriers, pharmaceutical carriers, thermal binders or other excipients. A particular example may be a mixture of the active pharmaceutical ingredient with a surfactant or surfactants, the active pharmaceutical ingredient with a polymer or polymers, or the active pharmaceutical ingredient with a combination of a surfactant and polymer carrier or surfactants and polymer carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a pharmaceutical composition of the present disclosure compared to peak solubility of the reference standard agent under the same conditions. Preferable, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of the active pharmaceutical ingredient that enhance bioavailability may comprise a mixture of the active pharmaceutical ingredient and one or more pharmaceutically acceptable adjuvants that enhance the bioavailability of the active pharmaceutical ingredient. Examples of such adjuvants include but are not limited to enzymes inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cₘₐₓ or the AUC of the active pharmaceutical ingredient as determined during in vivo testing, where Cₘₐₓ is the highest reached blood level concentration of the active pharmaceutical ingredient over time of monitoring and AUC is the area under the plasma-time curve. Enhanced bioavailability can be represented as the ratio of Cₘₐₓ or the AUC of the active pharmaceutical ingredient in a pharmaceutical composition of the present disclosure compared to Cₘₐₓ or the AUC of the reference standard the active pharmaceutical ingredient under the same conditions. This Cₘₐₓ or AUC ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

In some aspects, the amount of the excipient in the pharmaceutical composition is from about 0.5% to about 20% w/w, from about 1% to about 10% w/w, from about 2% to about 8% w/w, or from about 3% to about 7% w/w. The amount of the excipient in the pharmaceutical composition comprises from about 0.5%, 1%, 1.5%, 2%, 2.5%, 3%, 3.5%, 4%, 4.5%, 5%, 5.5%, 6%, 6.5%, 7%, 7.5%, 8%, 9%, to about 10% w/w, or any range derivable therein, of the total pharmaceutical composition. In one embodiment, the amount of the excipient in the pharmaceutical composition is at 4% to 6% w/w of the total weight of the pharmaceutical composition.

### II. HIGH ENERGY AND FUSION MANUFACTURING METHODS

In some aspects, the pharmaceutical formulations described herein are processed in a high energy mixing process, wherein the high energy mixing process is a process that utilizes one, two or more mixing blades in a thermokinetic chamber. The process may be described as involving mixing blades rotating at a high speed, measured in revolutions per minute, generally at a minimum speed of 500 RPM, depending on the size of the processing unit and the composition being processed. In one example, the high energy mixing process may further not comprise the use of an external heating source. Some non-limiting examples of high energy mixing process may include those which use a thermokinetic chamber such as those disclosed in U.S. Pat. No. 8,486,423, which is incorporated herein by reference. This disclosure is directed to a method of blending certain heat sensitive or thermolabile components in a thermokinetic mixer by using multiple speeds during a single, rotationally continuous operation on a batch containing thermolabile components in order to minimize any substantial thermal degradation, so that the resulting pharmaceutical compositions have increased bioavailability and stability. High energy mixing processes may be particularly useful for compounding heat-sensitive or thermolabile components. One advantage of the high energy mixing processes that may be used is that the method provides brief processing times, low processing temperatures, high shear rates, and the ability to compound thermally incompatible materials.

High energy mixing processes may be carried out in a thermokinetic chamber using one or multiple speeds during a single, compounding operation on a batch of components to form a pharmaceutical formulation described herein. The methods may further comprise one or more set speeds form about 500, 750, 1000, 1250, 1500, 1750, 2000, 2250, 2500, 2750, 3000, 3250, 3500, 3750, 4000, 4250, 4500, 4750, 5000, 5250, 5500, 5750, 6000, 6250, 6500, 6750, 7000, to about 7200 revolutions per minute (rpm) or any range derivable therein. The set speed may be from about 500 to about 7200 rpm, from about 1000 to about 6000 rpm, or from about 2000 to about 5000 rpm. In one example, the thermokinetic chamber may include a chamber having an inside surface and a shaft extending into or through the chamber. Extensions extend from the shaft into the chamber and may extend to near the inside surface of the chamber. The extensions are often rectangular in cross-section, such as in the shape of blades, and have facial portions. During high energy mixing processes, the shaft is rotated causing the components being compounded, such as particles of the components being compounded, to impinge upon the inside surface of the chamber and upon facial portions of the extensions. The shear of this impingement causes comminution, frictional heating, or both of the components and translates the rotational shaft energy into heating energy. Any heating energy generated during high energy mixing processes is evolved from the mechanical energy input. High energy mixing processes are carried out without an external heat source. The thermokinetic chamber and components to be compounded are not pre-heated prior to commencement of the high energy mixing processes. The thermokinetic chamber may include a temperature sensor to measure the temperature of the components or otherwise within the thermokinetic chamber. During the mixing process, the pharmaceutical composition may exhibit two distinct temperature profiles (also called regions) based upon the energy input during the mixing. The first period is a glass transition inflection region wherein the temperature of the composition being processed starts at room temperature then increases to a first maximum temperature followed by reducing to a first minimum temperature. At this minimum temperature at the end of the first period then the temperature rises to a relatively constant temperature to begin the region of prolonged mixing. This higher temperature after the glass transition inflection region is a region of prolonged mixing in which the temperature remains relatively constant. The temperature at the region of prolonged mixing has a constant temperature with a variation of less than about 10%, more preferably less than about 5% over time at this region. During this time period, the temperature may not change by more than about 20° C., 15° C., 14° C., 12° C., 10° C., 9° C., 8° C., 7.5° C., 7° C., 6° C., 5° C., 4° C., 3° C., 2° C., or less than about 1° C. These two regions can be readily seen when the temperature is plotted against mixing time. These two regions may be separated by a time period and the time period may be from about 0.5 second to about 5 minutes. In some embodiments, the time period is less than about 5 minutes, less than about 1 minute, less than about 30 seconds, less than about 20 seconds, less than about 10 seconds, or less than about 5 seconds. For comparison to a high energy mixing process known in the are not containing a TCE, at the minimum temperature at the end of the first period, the temperature rises and continues to rise until the ejection temperature is reached and does not exhibit a period of relatively constant temperature in a region of prolonged mixing.

The average maximum temperature in the thermokinetic chamber during high energy mixing processes may be less than the glass transition temperature, melting point, or molten transition point, of APIs present, one or all excipients, or one or all other components of the amorphous solid dispersion, or any combinations or sub-combinations of components. In some embodiments, the methods comprise reaching the desired specific elevated temperature at which mixing can occur, in the region of prolonged mixing. This specific elevated temperature is reached at least 30%, 20%, 10%, 5%, 1% quicker than a pharmaceutical composition which does not contain a thermally conductive excipient.

High energy mixing processes may be performed in batches or in a semi-continuous fashion, depending on the product volume. When performed in a batch, semi-continuous, or continuous manufacturing process, each high energy mixing processes step may occur for a time of less than 5, 10, 15, 20, 25, 30, 35, 40, 45, 50, 55, 60, 70, 100, 120, 240, or 300 seconds, or may be from about 5 second to about 300 seconds, or any range derivable therein.

Variations of high energy mixing processes may be used depending on the amorphous solid dispersion and its components. For example, the thermokinetic chamber may be operated at a first speed to achieve a first process parameter, then operated at a second speed in the same high energy mixing processes process to achieve a final process parameter. In other examples, the thermokinetic chamber may be operated at more than two speeds, or at only two speeds, but in more than two time internals, such as at a first speed, then at a second speed, then again at the first speed. Additionally, compositions of the present disclosure may be processed using any high energy technique known to one skilled in the art to produce a solid formulation, including any composition made by a high energy mixing process with no external heat added. In some embodiments, of these

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

A “high energy mixing process” refers to a formulation process that uses mixing blades rotating at a high speed, measured in revolutions per minute, generally at a minimum speed of 500 RPM, depending on the size of the processing unit and the composition being processed. This process may further comprise no heat applied from an external source.

The term “degradation” or “chemically sensitive” refers to a compound that is destroyed or rendered inactive and unacceptable for use. Degradation may include compounds which have one or more chemical bonds present in the compound has been broken.

The term “dissolution” as used herein refers to a process by which a solid substance, such as the active ingredients or one or more excipients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

A “poorly soluble drug” refers to a drug which meets the requires of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of solute, a sparingly soluble drug is a drug which is dissolved from 30 to 100 part of solvent required per part of solute, a slightly soluble drug is a drug which is dissolved from 100 to 1,000 part of solvent required per part of solute, a very slightly soluble drug is a drug which is dissolved from 1,000 to 10,000 part of solvent required per part of solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “essentially free of” or “essentially free” is used to represent that the composition contains less than 1% of the specific component. The term “entirely free of” or “entirely free” contains less than 0.1% of the specific component.

As used herein, the term “substantially intact” in terms of a specified component, is used herein to mean that the specified component has not been degraded or rendered inactive in an amount less than 5%. The term “essentially intact” is used to represent that less than 2% of the specific component has been degraded or rendered inactive. The term “entirely intact” contains less than 0.1% of the specific component has been degraded or rendered inactive.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferable less than 0.1 μm. In one embodiment, the composition is so homogeneously mixed in such a manner that there are no atoms of the thermally conductive excipient are adjacent to another atom of the thermally conductive excipient.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23° C., unless otherwise noted. An elevated temperature is a temperature which is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active agent to a patient in a single administration. Such unit dose formulations that may be used include but are not limited to a single tablet, capsule, or other oral formulations, or a single vial with a syringeable liquid or other injectable formulations. The resulting product can then undergo further downstream processing to create an intermediate product, such as granules, that can then be further formulated into an unit dose such as one prepared for oral delivery as tablets, capsules, three dimensionally printed selective laser sintered (3DPSLS) or suspensions; pulmonary and nasal delivery; topical delivery as emulsions, ointments or creams; transdermal delivery; and parenteral delivery as suspensions, microemulsions or depot. In some forms, the final pharmaceutical composition that is produced is no longer a powder and is further produced as a homogenous final product. This final product has the capability of being processed into granules and being compressed or 3DPSLS into a final pharmaceutical unit dose form.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1— Methods and Materials

### A. Materials

Candurin® gold sheen was purchased from EMD Performance Materials (Philadelphia, Pa.). AQOAT® Hypromellose acetate succinate HMP grade (HPMCAS-HMP) was donated by Shin-Etsu Chemical Co., Ltd. (Tokyo, Japan). Boehringer Ingelheim (BI) research compound BI639667 (BI-667) was donated by BI (Ingelheim, Germany). Glass number 50 capillary (2.0 mm) was purchased from Hampton Research Corp. (Aliso Viejo, Calif.). HPLC grade acetonitrile, methanol and Trifluoracetic acid (TFA) were purchased from Fisher Scientific (Pittsburgh, Pa.). Monohydrate and dihydrate sodium phosphate salts were purchased from Fisher Scientific (Pittsburgh, Pa.). Fasted state simulated intestinal fluid (FaSSIF) powder was purchased from Biorelevant.com Ltd (Surrey, United Kingdom).

### B. Characterizing TCE Interactions

1. Modulated Differential Scanning Calorimetry

Modulated differential scanning calorimetry (mDSC) was conducted on a Q20 DSC unit (TA Instruments, New Castle, Del.). 8-10 mg of sample was weighed with a Sartorius 3.6P microbalance (Gottingen, Germany) into standard aluminum pans and covered with a standard aluminum lid. Thermal analysis was performed with a nitrogen sample purge of 50 mL/min. Measurement parameters for detecting changes in melting point (Tₘ) and glass transition (T_{g}) when the TCE is incorporated were determined using a heating rate of 3°/min from 35-220° C. with a modulation of 0.3° C. every 50 seconds.

2. Fourier-Transform Infrared Spectroscopy

Interaction between the TCE and other components in the composition were evaluated using Attenuated total reflectance Fourier-transform infrared spectroscopy (ATR-FTIR) on a Nicolet™ iS™ 50 spectrometer (Thermo Scientific, Waltham, Mass.). Measurements were performed using a germanium crystal that supplied constant torque during the analysis. Analysis conditions scanned a range of 700-4000 cm⁻¹ using a resolution of 4 cm⁻¹ with 64 scans. Results were evaluated using OMNIC™ analysis software.

3. Hot Stage Polarized Light Microscopy

Hot stage polarized light microscopy (HSPLM) and polarized light microscopy (PLM) were used to determine amorphous nature and visual interactions between excipients when melting. Samples were prepared by lightly spreading physical mixtures of the two compositions onto a glass slide. An Olympus BX60 microscope (Olympus Corp., Center Valley, Pa.) with Insight QE camera (Diagnostic Instruments, Inc., Sterling Heights, Mich.) was used for visual observation. A FP 90 central processer controlled the FP82HT hot stage (Mettler Toledo, Columbus, Ohio) was used to heat the state to a temperature of 130° C. for 15 minutes followed by 210° C. for 15 minutes. Images were captured using Spot Advance Software (Diagnostic Instruments, Inc.).

C. Sample Processing

1. KinetiSol Processing and Milling

Physical mixtures of two compositions of BI-667:HPMCAS-HMP (1:2) and BI-667:Candurin:HPMCAS-HMP (33:3:64) were prepared by mixing in plastic bags for 5 minutes. KinetiSol Processing was performed on a KinetiSol research formulator, KBC20 (DisperSol Technologies, LLC, Georgetown, Tex.). A 15 gram batch size was used for all compositions. An initial mixing stage of 500 rpm for 5 seconds was used to ensure homogenous mixing for all batches. The different compositional processing parameters are expressed in Table 1. KinetiSol formulations can either be processed at a set RPM and ejected at a desired temperature or processed at a set RPM and ejected after a defined period of time. An optic fiber IR probe was used to measure sample temperature as it changed from the frictional and shear forces applied to the system. All samples were manually quenched between two metal plates. The Quenched sample was placed in a disposable MT 40 grinding chamber and milled using an IKA tube mill control (IKA-Werke, Staufen, Germany) operated at 20,000 RPM for 30 seconds with a 15 second pulse. The resulting particles were passed through Advantech sieves to only isolate particles <125 μm for analysis. The milled sample composition of BI-667:HMP (1:2) is hereafter referred to as KSD. The milled sample composition of BI-667:Candurin:HMP (33:3:64) is hereafter referred to as KSD-TCE. The first numeric values following the formulation name corresponds to the processing time and the second numeric values corresponds to the RPM (e.g., KSD-TCE-60-3400). For example, KSD-TCE-60-3400, would represent a KinetiSol Processed ternary composition containing candurin that was processed for 60 seconds at 3400 RPM. Sample compositions, processing conditions and processing profiles can be found in Table 2.

### D. Final Composition Characterization and Performance Evaluation

1. Powder X-Ray Diffraction

Powder X-ray diffraction (PXRD) was performed on a Rigaku MiniFlex600 (Rigaku, The Woodlands, Tx, USA) that utilized a Cu-Kα radiation source operated at a voltage of 40 kV and a current of 15 mA. Powder samples were dispensed in aluminum sample holders. The method parameters for analysis scanned a two-theta range of 10-35° with a scan speed of 2.0°/min, step size of 0.02° while rotating the sample. Data analysis was performed using MDI JADE 9 software (Materials Data Inc., Livermore, Calif.).

2. Wide-Angle X-Ray Scattering

WAXS measurements used a custom-built SAXSLab instrument (SAXSLab, Northampton, Mass., USA) at the University of Texas at Austin (Austin, Tex., USA). The instrument is equipped with a microfocus Cu k-alpha rotating anode X-ray source operated at 50 kV and 0.6 mA and a PILATUS3 R 300K (DECTRIS Ltd., Philadelphia, Pa., USA) detector. The detector is equipped with three detecting modules of 83.8×106.6 mm² sensitive area. The pixel size is 172×172 μm². The distance between the sample and detector ranged from 0.95 to 1.45 m. Disposable glass capillaries (Hampton Research, Aliso Viejo, Calif., USA) of a 2.0 mm outside diameter were used to load samples. Ganesha instrument control center software (SAXSLab, Northampton, Mass., USA) was used to control the instrument. The configuration of 2 apertures WAXS and 2 mm off-centered beam stop was used for all measurements. The acquisition time for each sample was set at 300 s with a beam stop mask and correction for the sample thickness of 2.0 mm. All data were corrected for cosmetic background radiation and an incident beam strength by measuring the X-ray intensity directly on the detector. Data analyses were performed using SAXSGUI software (SAXSLab, Northampton, Mass., USA).

3. High Pressure Liquid Chromatography

A Thermo Scientific Dionex UltiMate 3000 high pressure liquid chromatography (HPLC) system (Thermo Scientific, Sunnyvale, Calif.) equipped with Chromeleon 7 software was used for in vitro dissolution and purity potency analysis. The HPLC method detailed in [Jermain et al] was adapted for sample analysis. Briefly, the aqueous phase, Mobile phase A, consists of 0.05% TFA in deionized water. The organic phase, mobile phase B, consists of 0.05 TFA in acetonitrile. During analysis the system is held isocratic (80% A, 20% B) for 5 minutes, a gradient is then initiated from 5 minutes to 15 minutes (80% A→20% A). From 15 minutes to 16 minutes a second gradient was ran (80% A→5% A) then a third gradient was run from 16 minutes to 17 minutes (5% A→20% A) and lastly held isocratic (80% A, 20% B) from 17 minutes to 20 minutes. The method is able to separate out all 3 impurities related to the BI-667 compound. The flow rate is set to 1 mL/min with a 20 minute run time and a 10 μL injection volume. Linearity was achieved from 1-200 μg/mL with a retention time of 9.0 minutes. An UltiMate RS Variable Wavelength detector was set to 225 nm. The column used for separation was a Luna® C18, 3 μm, 3.0 mm×100 mm, P/N: (Phenomenex®, Torrance, Calif.).

4. Non-Sink pH-Shift Dissolution

Final pharmaceutical dosage forms were made using size 2, HPMC based capsules. Each capsule incorporates 120 mg KSD (80%) and 30 mg (20%) of anhydrous lactose as a diluent. Attention was taken to ensure all samples had the same particle size distribution for the KSD material, PSD<125 μm. A small volume pH shift dissolution with biorelevant media was employed to mimic gastrointestinal transit of orally administered capsules. Dissolution was performed in a SR8 Plus dissolution tester (Hanson Research Corp., Chatsworth, Calif.) equipped with minipaddles and 150 mL glass vessels operated at a temperature of 37° C. and a paddle speed of 100 rpm. The vessels initially contained 90 mL of 0.01N HCL and at 30 minutes 60 mL of Fassif (2.24 g/L SIF in 0.1 M sodium phosphate buffer, pH 6.8) was added to each vessel to make a total volume of 150 mL. 1 mL samples were taken, immediately filtered through 0.22 μm, 13 mm PES syringe filters, and diluted 1:1 with 95:5 methanol:deonized water. An equivalent amount of media was replaced at all time points: 5, 10, 15, 25, 35, 45, 60, 90, 120, 180, 240, and 360 minutes. The pH was measured at the conclusion of the study to ensure a pH of 6.8 was maintained for all samples. All samples were performed in triplicate (n=3) and BI-667 concentration was determined using the aforementioned HPLC method.

### Example 2—Results of Formulations with Thermally Conductive Excipients

### A. Characterizing TCE Interactions

Preformulation characterization of the TCE's, candurin, interaction with the individual components and in combination were investigated. Thermal analysis with mDSC was used to detect any change in the glass transition or melting temperature of the components when candurin was incorporated. mDSC results shown in Table 1, indicate no change in glass transition or melting temperature when candurin is incorporated.

TABLE 1
mDSC analysis to identify potential change in
glass transition or melting point when the thermally
conductive excipient, candurin, is incorporated.

| | HPMCAS- | BI-667 | HPMCAS- |
|---|---|---|---|
| | HMP | Tₘ | HMP:BI667 |
| | T_{g,mid} (° C.) | (° C.) | Tₘ (° C.) |
| No candurin | 122.04 | 206 | 194.62 |
| 3% candurin | 122.64 | 205.2 | 195.72 |

mDSC findings were confirmed by directly determining interactions at a molecular level with FTIR, results shown in FIG. 2. Candurin shows no relevant presence within the spectra observed. No changes in molecular interaction of the individual components or in combination is observed when candurin is present.

The aforementioned solid-state characterization techniques confirm no molecular interactions between candurin and compositional components. HSPLM was performed to observe the impact of candurin when the composition is in a molten state, FIG. 3. HPMCAS-HMP exhibits birefringence when observed under polarized light microscopy, areas of crystallinity related to the polymer are identified. The HPMCAS-HMP crystals remained unchanged throughout the experiment, whereas BI-667 melted at 210° C. No difference in the solubilization of the BI-667 compound is observed when candurin is present. At all studied temperature ranges it can be observed that no physical change in candurin occurs.

### B. KinetiSol Processing

Controlling processing parameters between compositions allows for a direct comparison of candurin's effect on KinetiSol processing. Mixing dynamics vary depending on the sample size studied, attention was taken to ensure all compositions processed were 15 grams. The reference name with respective processing conditions are given in Table 2.

TABLE 2
KinetiSol Processing Parameters and Composition Components

| | | | Ejection | | Prolonged | XRD |
|---|---|---|---|---|---|---|
| Reference | Time | | Temp | Candurin | mixing achieved | Amorphous |
| Name | (s) | RPM | (° C.) | (3%) | (Y/N) | (Y/N) |
| KSD-TCE-25-3400 | 25 | 3400 | 76.1 | Y | Y | N |
| KSD-TCE-35-3400 | 35 | 3400 | 128.9 | Y | Y | Y |
| KSD-TCE-45-3400 | 45 | 3400 | 130.5 | Y | Y | Y |
| KSD-TCE-65-3400 | 65 | 3400 | 133.4 | Y | Y | Y |
| KSD-TCE-92-2700 | 92 | 2700 | 119.4 | Y | Y | Y |
| KSD-54-3400 | 54 | 3400 | 160 | N | N | Y |
| KSD-25-2700 | 25 | 2700 | 135 | N | N | N |
| KSD-20-5000 | 20 | 5000 | 160 | N | N | Y |

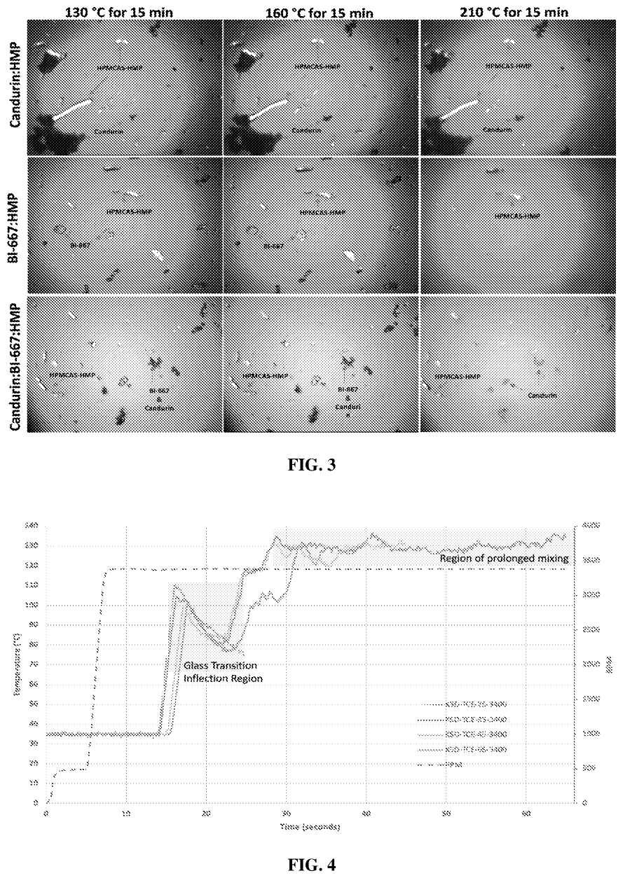

A comparison of the temperature change over time was investigated with candurin containing samples held at a fixed 3400 RPM, FIG. 4. For these samples an ejection temperature was not registered, but instead processed based on a selected time interval. As the processing interval in which the KSD-TCE samples were increased the compositions temperature remained constant across all time intervals. This confirms candurin's ability to dissipate energy input from the mixing elements into the KinetiSol chamber. Additionally, in this comparison, a time dependence that corresponds to an optimal mixing interval in order to produce amorphous samples is observed.

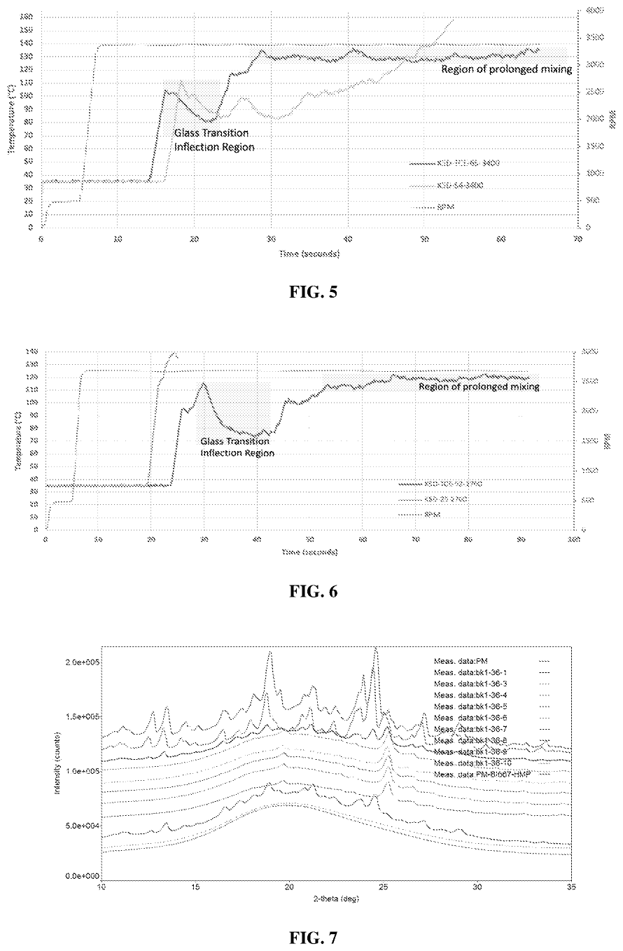

Previous publications using the KinetiSol process rely on the process's capability of rapidly mixing and minimizing sample exposure at elevated temperatures. In these examples, regardless the constituents of the composition, when energy is applied, the composition temperature increases at a rate relative to the energy input into the system (Hughey et al., 2011; LaFountaine et al., 2016b; DiNunzio et al., 2010b; Ellenberger et al., 2018b). Previously, a steady-state processing, where the composition can maintain prolonged mixing at a fixed temperature, had not been achieved. FIG. 5, compares the prolonged mixing achieved with a KSD-TCE composition to the profile of a KSD composition. All compositions were processed under the same conditions, the only variable was the presence of candurin. The KSD-54-3400 curve yielded a steady increase in temperature after the glass transition inflection region and did not experience a region of prolonged mixing. The composition was ejected as the deviation in processing temperatures became apparent. Divergence in processing profiles of the KSD-54-3400 and KSD-TCE-65-3400 compositions appear after the glass transition inflection region. The ability of candurin to modify the thermal conductive properties of the composition is shown two-fold in FIG. 6. First, the ability of the KSD-TCE-65-3400 formulation to dissipate the energy input from the mixing elements to the KinetiSol chamber and achieve a steady state is highlighted by the ability to maintain a region of prolonged mixing. The improved thermal properties not only enable the composition to dissipate heat more efficiently but also allows the composition to conduct heat more efficiently. This can be seen from the discrepancy in time that it takes each formulation to reach 125° C., the onset region of prolonged mixing in this example. By the time the KSD-54-3400 composition reaches 125° C., the KSD-TCE-65-3400 composition had been mixing at a steady state for 20 seconds.

Hughey et al. postulated that in an optimized system, composition ejection would take place at the glass transition temperature to avoid excessive thermal energy input (Hughey et al., 2011). In the system studied, the experimental glass transition value, 122° C., of HPMCAS-HMP is in alignment of that reported from the manufacture and previous literature (Meena et al., 2016; Friesen et al., 2008). In FIG. 6, the RPMs are decreased from 3400 to 2700. The TCE composition still maintains the same capacity to transfer energy, but with less energy input the temperature at which prolonged mixing occurs is decreased. KSD-TCE-92-2700 is processed at the glass transition temperature of the composition for a prolonged interval, the compositional temperature never surpasses 122° C. and produces an amorphous composition. KSD-25-2700 rapidly increases in temperature and is ejected at 135° C. This composition is processed at a higher temperature but does not produce an amorphous composition. Hughey et al. (2011) also observed that as the processing speed is decreased the inflection point becomes more apparent (Hughey et al., 2011). This statement holds true for the TCE compositions processed in FIG. 7.

### C. Final Composition Characterization and Performance Evaluation

KSD and KSD-TCE formulations were characterized by solid-state characterization techniques to determine the amorphous nature of the composition. An overlay of the pXRD analysis is shown in FIG. 8. Candurin has many Bragg's peaks across the two-theta region analyzed, with the most intense being at 19.8 and 25.2 two-theta degrees. BI-667 exhibits Bragg's peaks at 18.9, 21.5, 23.8 and 24.7 two-theta degrees (Jermain et al., 2019). Expected amorphous halos without peaks are not detected for samples containing the TCE, with Bragg's peaks seen at 19.8 and 25.2 two-theta degrees. Natural candurin and BI-667 are incorporated into the overlay to exemplify the peaks present in the KSD-TCE samples correspond to candurin and not crystalline BI-667.

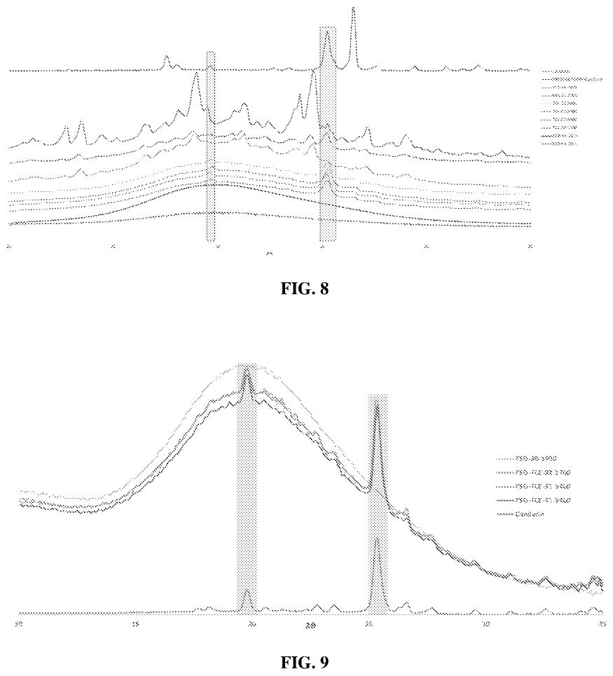

Confirmation of the pXRD results were confirmed using WAXS, an advanced solid-state characterization technique (Ma et al., 2020). The same characteristic peaks of candurin at 19.8 and 25.2 were present for the KSD-TCE samples and absent in KSD samples. All KSD compositions maintained the broad halo reported from pXRD analysis. WAXS Results are shown in FIG. 9.

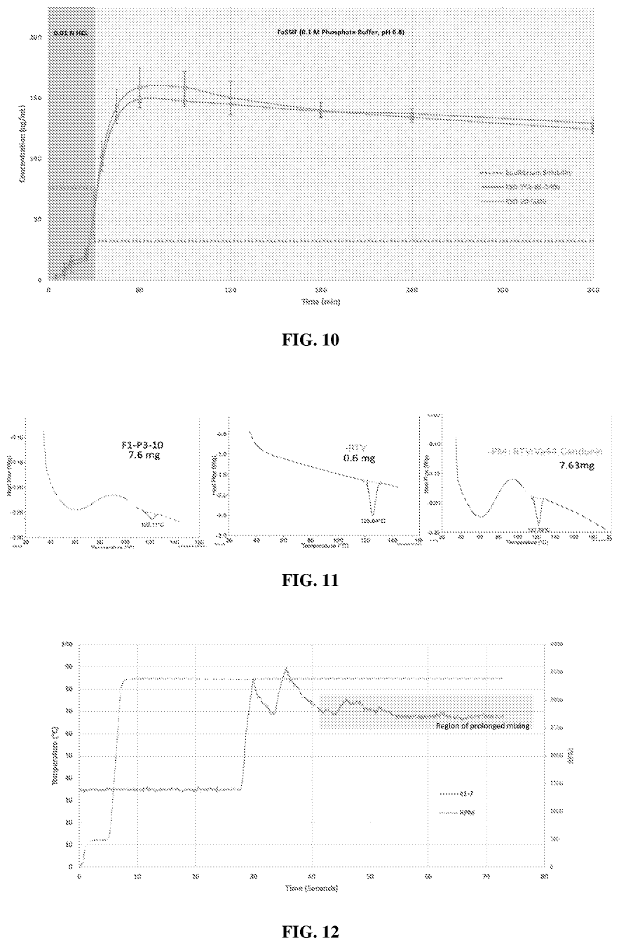

The aforementioned results exhibit the ability of candurin to modify KinetiSol processing profiles without molecularly interacting with the composition. The TCE's impact upon product performance when incorporated into the composition was evaluated using a small volume, non-sink, pH-shift dissolution study. The KSD-TCE capsule reflected the dissolution profile of the KSD capsule, shown in FIG. 10. Both compositions retarded drug release in the acidic phase and then rapidly released BI-667 upon pH shift to the neutral phase. All compositions were evaluated for purity and potency. HPLC analysis confirmed none of the processed compositions experienced degradation.

Incorporation of the TCE allows for this steady state to be achieved at less aggressive mixing conditions to minimize exposure to aggressive shear conditions and elevated temperatures. This allows for intimate mixing for extended intervals at time at a desired temperature. Purity and potency analysis were performed to ensure these extended mixing intervals were not increasing drug degradation. Degradation or a decrease in potency was not detected in any of the samples that had TCE incorporated and were mixed for an extended period of timing and various rpms.

### Example 3—Discussion of Pharmaceutical Compositions with Thermally Conductive Excipients

### A. Characterizing TCE Interactions

Molecular interactions can be indirectly identified by mDSC thermal analysis. A change in the systems glass transition or melting temperature can be described by the Flory-Huggins interaction parameter that accounts for Van der Waals forces, hydrogen bonding, charge transfer complexation and ionic interactions (Flory, 1953; Marsac et al., 2006). The melting point depression observed between BI-667 and HPMCAS-HMP, Table 1, is a result of a weak molecular interaction to form a drug polymer complex (Sarode et al., 2013; Meng et al., 2015). Incorporation of candurin into the system showed no change in the glass transition temperature or melting point of the compositions, thus supporting the absence of interaction of candurin with the system.

Thermal analysis results were investigated by FTIR to directly observe potential interactions at a molecular level not detectable by mDSC. No change in the spectra was observed for the physical mixture, individual components or processed compositions. FTIR was able to confirm the mDSC results and that no interaction is occurring at a molecular level between candurin and the composition. Based on previous literature, candurin's lack of interaction with the composition suggests the inability of candurin to provide an effect on the compositions solubility or the stability of the system (Sarode et al., 2013; Meng et al., 2015; Baird and Taylor, 2012).

Candurin exhibits excellent thermal stability as shown by the temperatures candurin was exposed to during this study that were mild in comparison to temperatures it is manufactured at, 900° C. Candurin also does not exhibit solubility in water or other common solvents (Folmer and Rao, 2013; and Evaluation of certain food additives and contaminants, F.a.a. organization and W.H. Organization, Editors). Therefore, it would not be expected to observe any physiochemical change during the formulation process. This is confirmed with visual observation from the HSPLM study. A physical mixture containing BI-667 and candurin were lightly spread over the polymer film and upon melting, the BI-667 crystals completely dissolve within the polymer leaving the candurin left unchanged. It is also notable to mention that when only heat is applied at 130 and 160° C., the heat alone is not sufficient to dissolve the BI-667 crystals. KinetiSol processing was able to produce ASDs at both 130 and 160° C. stressing that heat is not the only component responsible for the dissolution of drug into a molten polymer.

### B. Expanded KinetiSol Processing

The capacity at which candurin can modify KinetiSol processing was investigated. By eliminating candurin's potential benefit being attributed to molecular interactions, without wishing to be bound by any theory, it is believed that the benefit of candurin is credited to its ability to increase the thermal conductivity of the system. Polymers have low thermal conductivity, typically 0.1-0.6 (W/m K) (Yang, 2007), where metals can be an order of 3 orders of magnitude higher i.e. such as 237 (W/m K) for aluminum (Touloukian et al., 1970). Again, without wishing to be bound by any theory, it is believed that even incorporation of the slightest amount of candurin, an aluminum containing excipient, into the composition would improve the thermal conductive properties of the system. The system benefits from the increased thermal conductivity not only in the final product but during the process. By improving the ability of the polymer matrix to absorb and dissipate energy more efficiently, the process achieves prolonged mixing at a relatively constant temperature.

The implications of this benefit can be seen in FIG. 5. The composition containing the TCE quickly achieves a steady state after the glass transition inflection region and is then processed in the prolonged mixing region for the entirety of the experiment. The improved thermal conductivity creates an equilibrium where temperature is maintained due to consistent energy applied to the system by the mixing elements and improved heat dissipation from the composition to the KinetiSol chamber. The KSD composition without the TCE component steadily increases in temperature without leveling off. The change in the process is apparent, but the potential impact is not realized in this example. While FIG. 5 highlights the ability of candurin to modify the processing profile, both samples can produce a viable ASD with no degradation. To see a realized advantage of incorporating the TCE, drug molecules that are extremely sensitive to heat, such as meloxicam, could be processed below the degradation temperature for an extended interval of time.

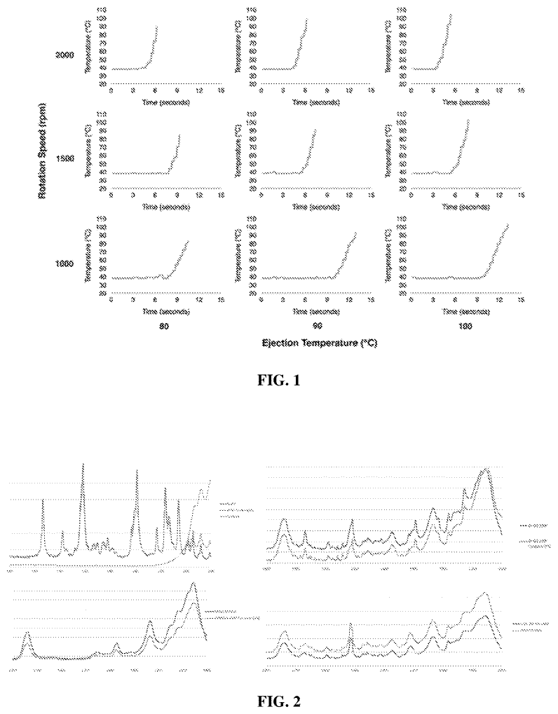

The benefit of processing for an extended time interval is highlighted by the time dependence necessary to achieve an amorphous solid dispersion, FIG. 4. Composition KSD-TCE-25-3400 was not able to mix for a sufficient time and crystalline BI-667 was detected in the composition after processing. As the mixing interval increased to 35 seconds and longer, complete conversion to the amorphous phase was achieved. In addition, FIG. 1 shows the relationship between mixing time and temperature to create amorphous solid dispersions. LaFountaine et al. (2016) was able to successfully formulate PVA 4-88 and ritonavir (30%) in an amorphous solid dispersion by mixing at temperatures above 40° C. for less than 3 seconds and ejecting before the glass transition inflection region (LaFountaine et al., 2016b). In a similar manner, KSD-25-2700 is processed for 5 seconds at elevated temperatures and ejected at 135° C. KSD-TCE-92-2700 never achieves a temperature greater than 122° C. but experienced prolonged mixing for ˜40 seconds. Despite the lower formulation temperature, the prolonged mixing, enabled by incorporation of the TCE, allows the KSD-TCE-92-2700 composition to produce an amorphous product, whereas crystallinity is detected in the KSD-25-2700 product.

The importance of the TCE in the composition to enable KinetiSol processing to mix at a relatively constant temperature is shown above. By comparing formulation KSD-TCE-54-3400 and KSD-TCE-92-2700 it was observed that as the RPMs are decreased, the ability to control the temperature at which the formulation is processed for extended time intervals is further understood. By decreasing the RPMs, the energy input into the system is decreased while the composition is still dissipating heat out of the system to the same extent. This results in a decrease in total energy to the system, resulting in a lower formulation temperature. Thoughtful consideration of the RPM selection can be used to optimize the temperature at which the prolonged mixing region occurs in order to meet the needs of each unique drug and system. Ideally, similarly to FIG. 7, prolonged mixing would occur at the polymers glass transition temperature to minimize any excessive exposure to elevated temperatures.

To better understand the benefit of prolonged mixing at a relatively constant temperature when applied to the KinetiSol Process, the mixing chamber can be described as a dissolution vessel and the mixing elements as the paddles. As the temperature at which the formulation is processed reaches the polymer's glass transition temperature, the molten polymer behaves as a viscous liquid. As a viscous liquid, the crystalline particles of drug will be dispersed into the molten polymer by the mixing elements of the KinetiSol equipment (Hughey et al., 2011). Dissolution parameters derived in the Noyes-Whitney equation may help explain solubilization of the crystalline particles of drug into the molten polymer, and this deserves further exploration.

$$\frac{dm}{dt} = A \frac{D}{d} ( {C}_{s} - {C}_{b} )$$

The Noyes-Whitney equation is described by Equation 1. The parameters are: surface area (A), diffusion (D), diffusion layer thickness (d) and the gradient between the surface concentration and bulk concentration (Cₛ−C_{b}). The KinetiSol process embodies many of these parameters to which when applied to the composition can help explain crystalline drug solubilizing within the molten polymer.

BI-667 was observed to not dissolve in HPMCAS-HMP at either 130° C. or 160° C., which are both above the glass transition temperature of the polymer (122° C.), when no mixing was applied to the composition in HSPLM, FIG. 3. When the impact of mixing was considered in the KinetiSol process, crystalline particles of BI-667 were homogenously distributed throughout the molten polymer and subsequently dissolved to form a molecular solution of BI-667 within HPMCAS-HMP. As evidence, KSD-TCE-92-2700 is able to convert crystalline BI-667 to the amorphous state without surpassing the polymer's glass transition temperature.

Not wishing to be bound by any particular theory, particle mixing in a liquid is generally governed by three types of motion: molecular diffusion, eddy diffusion (turbulent flow) and convection (Hold, 1982). The Noyes-Whitney equation, which is derived from Fix's law, only models molecular diffusion (Fick, 1855). In viscous polymer systems, like those studied in this paper molecular diffusion and eddy diffusion are negligible, thus making convection primarily responsible for molecular movements (Hold, 1982). Therefore, convection as applied to compositions made by KinetiSol processing relates to the movement of drug particles throughout a molten viscous liquid (e.g. polymer), resulting in either distributive and/or dispersive mixing. The contribution of distributive mixing to the overall degree of mixing is not as significant as dispersive mixing, but it is essential in ensuring spatial homogeneity of the final composition (Grace, 1982; Shenoy, 1999). Distributive mixing of the drug particles throughout the molten viscous liquid during the KinetiSol process involves bulk motion of the composition from the pushing force of the mixing element. Dispersive mixing is achieved from both shear and elongational flow (Shenoy, 1999; Jia et al., 2014). The pitched mixing element (blade(s)) converges the composition's flow, forcing the composition through the narrow gap between the mixing element and the chamber wall (Jia et al., 2014; Brough et al., 2013). This elongational flow further mixes the composition and provides discrete particles that have the highest surface area possible of the crystalline drug to increase the dissolution to an extent it will dissolve at the solubility limit at the related temperature (Jia et al., 2014; Rauwendaal, 1999). KinetiSol processing utilizes both distributive and dispersive mixing to maximize the solubilization of the drug within the polymer melt to form the amorphous solid dispersion.

As discussed, temperature is not the only component increasing drug solubilization in the polymer; KSD-TCE-92-2700 elucidates the contribution of mixing to create a molecular dispersion. The intimate mixing accomplished by KinetiSol processing minimizes the diffusion gradient, d, through which the solute molecules must travel to reach the bulk solution. As the diffusion gradient is minimized, the dissolution rate is maximized. Furthermore, the viscosity and the RPMs influence the distributive and dispersive mixing ability of the KinetiSol process. Increasing the degree of mixing ensures the solubilization will only be limited by the saturation solubility of the crystalline drug within the polymer melt at that temperature. TCEs enable KinetiSol processing of compositions at a fixed temperature for a prolonged interval. Increasing the mixing interval allows for the distributive and dispersive mixing to further work to solubilize the crystalline drug into the polymer melt. As a result, longer mixing intervals can be used to process the composition at lower temperatures which can be extremely beneficial for thermally labile compounds.

### C. Evaluating TCE Impact on Performance

Pre-formulation characterization excluded the potential interaction between candurin and the compositional components. Candurin benefits the composition by increasing thermal conductivity to an extent that energy can dissipate out of the system rapidly enough to achieve a steady state where prolonged mixing at a desired temperature is possible. The presence of the candurin homogenously distributed within the final sample warrants further investigation on the impact on product performance.

Based on the work of Jermain et al. who processed BI-667:HPMCAS-MMP (1:2) blends at 4500 RPM for 22 seconds to generate ssNMR confirmed amorphous solid dispersions similar conditions (5000 RPM and 20 seconds) were adapted for the control, KSD-20-5000. The candurin containing comparator, KSD-TCE-92-2700, was exposed to the longest processing time, lowest temperature and least aggressive mixing conditions. KSD-20-5000 and KSD-TCE-92-2700 were compared by FTIR, WAXS and small volume, pH shift dissolution with no differences detected. Additionally, the enteric nature of HPMCAS-HMP was preserved after exposure to prolonged processing of KSD-TCE-92-2700, represented in FIG. 10. The candurin containing sample showed no difference in product performance when compared to that of the control.

Unlike plasticizers and surfactants, incorporation of the TCE does not positively or negatively impact the performance of the ASD (Hancock et al., 1995; Vasconcelos et al., 2007). This lack of affect can be attributed to the TCE not interacting with the composition at a molecular level, FIGS. 1 & 2. The TCE acts in principle to improve heat transfer.

### Example 4—Other Processing Methods

A sample was made according to the parameters and compositions described in U.S. Patent App. Pub. No. 2019/037441. Ritonavir, a poorly water-soluble drug that is shear and heat labile, was used as the drug, VA64 as a polymer and candurin, the thermally conductive excipient, in a ratio of 10:87:3 by weight, respectively. The printing parameters described are: Surface temperature 0-200° C. preferably 70-170° C., Chamber temperature 25-200° C. preferably 60-150° C., Layer thickness 10 mm-0.01 mm, beam size 0.0025-1 mm, scan speed 5 mm/s to 50,000 mm/s preferably 20-300 mm/s, Laser power 0.5 W to 140 W preferably 1.7-8 W, and wavelength 200 nm to 11,000 nm. In this reference example, a surface temperature between 100-110° C., chamber temperature of 90° C., Layer thickness of 0.1 mm, beam size of 0.25 mm, Laser scan speed of 25 mm/s, Laser power of 2.3 W, and wavelength of 445 nm were used. Table 3 identifies printing parameters used from U.S. Patent Application Publication No. 2019/037441 and these compositions were made as a tablet, but it was not an amorphous solid dispersion as each composition either could not be formulated or contained some degree of crystallinity. See FIG. 11 which shows that each of these formulations resulted in some crystallinity and could not produce an amorphous formulation.

TABLE 3
Printing Parameters

| Formulation | % | | S.T. | C.T. | | |
|---|---|---|---|---|---|---|
| Key | RTV | L.S. | (° C.) | (° C.) | H.S. | Comments |
| F1-P1-10 | 10 | 25 | 110 | 90 | 125 | Tablet could |
| | | | | | | not be made |
| F1-P2-10 | 10 | 25 | 110 | 90 | 25 | Tablet could |
| | | | | | | not be made |
| F1-P3-10 | 10 | 2S | 100 | 90 | 25 | Tablet had |
| | | | | | | crystallinity |
| | | | | | | present |

### Example 5

To demonstrate applicability to other drug-polymer systems, the TCE was incorporated into a Soluplus®-carbamazepine matrix. Ten grams of the formulation, 41-7, was processed at 500 RPM for 5 seconds and then processed at 3400 RPM for 68 seconds, FIG. 12. Similar to the previous examples, the TCE enables prolonged mixing to occur after the glass transition inflection region. The prolonged mixing processing temperature was lower due to the polymer's lower glass transition temperature (i.e., 70° C.). Processing at 3400 RPM allowed for prolonged mixing at the polymers glass transition temperature for over 30 seconds before the formulation was manually ejected at 68° C. During processing, the prolonged processing window's temperature did not vary by more the 5° C. during, ranging from 73° C. to 68° C. The composition of formulation 41-7 is 20% carbamazepine, 77% Soluplus®, and 3% candurin.

### Example 6

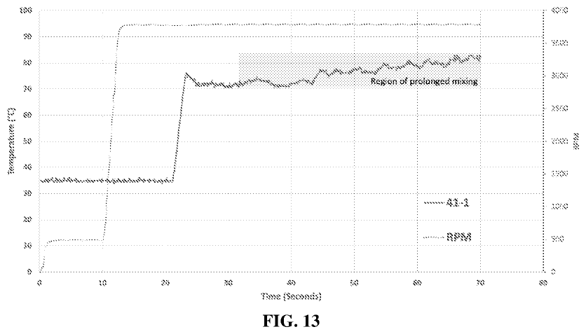

The following example demonstrates the ability to achieve prolonged mixing in the absence of an API. The processed composition 41-1 consists of 97% Soluplus® and 3% candurin. Candurin aids in heat dissipation to allow for prolonged mixing of Soluplus®. Unlike example 5, formulation 41-1 is processed at a higher RPM, 3800 RPM, compared to 3400 RPM. This increased RPM creates a region of prolonged mixing at a slightly higher temperature compared to example 5, highlighting the ability to control the processing temperature for prolonged periods, FIG. 13. In this example, the formulation doesn't change more than 10° C. during prolonged processing. For processing 10 grams of formulation 41-1 were processed at 500 RPM for 10 seconds and then 3800 RPM for 60 seconds.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Patent App. Pub. No. 2006/0084742
U.S. Patent App. Pub. No. 2019/037441
U.S. Pat. No. 5,900,447
U.S. Pat. No. 9,434,870
Alexy et al., Polymer Degradation and stability, 85(2): p. 823-830, 2004.
Baird and Taylor, Advanced drug delivery reviews, 64(5): p. 396-421, 2012.
Bergstrom et al., Advanced drug delivery reviews, 101: p. 6-21, 2016.
Brough et al., AAPS PharmSciTech, 17(1): p. 167-179, 2016a.
Brough et al., AAPS PharmSciTech, 17(1): p. 180-190, 2016b.
Brough et al., Thermo-kinetic mixing for pharmaceutical applications. Google Patents, 2013.
DiNunzio et al., European journal of pharmaceutical sciences, 40(3): p. 179-187, 2010a.
DiNunzio et al., European journal of pharmaceutics and biopharmaceutics, 74(2): p. 340-351., 2010b.
Ellenberger et al., AAPS PharmSciTech, 19(5): p. 1933-1956, 2018a.
Ellenberger et al., AAPS PharmSciTech, 19(5): p. 1985-1997, 2018b.
Evaluation of certain food additives and contaminants, F.a.a. organization and W.H. Organization, Editors.
Fick, The London, Edinburgh, and Dublin Philosophical Magazine and Journal of Science, 10(63): p. 30-39, 1855.
Fina et al., International journal of pharmaceutics, 529(1-2): p. 285-293, 2017.
Fina et al., International journal of pharmaceutics, 541(1-2): p. 101-107, 2018.
Flory, Principles of polymer chemistry. Cornell University Press, 1953.
Folmer and Rao, Potassium Aluminium Silicate-Based Pearlescent Pigments, Types I, II and III. Chemical and Technical Assessment (CTA), 2013.
Friesen et al., Molecular pharmaceutics, 5(6): p. 1003-1019, 2008.
Grace, Chemical Engineering Communications, 14(3-6): p. 225-277, 1982.
Gupta et al., AAPS PharmSciTech, 17(1): p. 148-157, 2016b.
Gupta et al., Journal of Excipients and Food Chemicals, 5(1): p. 1001, 2016a.
Hancock et al., Pharmaceutical research, 12(6): p. 799-806, 1995.
Handbook of Pharmaceutical Salts: Properties, and Use (P. H. Stahl & C. G. Wermuth eds., Verlag Helvetica Chimica Acta, 2002).
Hold, Advances in Polymer Technology: Journal of the Polymer Processing Institute, 2(2): p. 141-151, 1982.
Hughey et al., AAPs Pharmscitech, 11(2): p. 760-774, 2010.
Hughey et al., International journal of pharmaceutics, 419(1-2): p. 222-230, 2011.
Jermain et al., Drug development and industrial pharmacy, 45(5): p. 724-735, 2019.
Jermain et al., International journal of pharmaceutics, 535(1-2): p. 379-392, 2018.
Jia et al., Polymer composites, 35(5): p. 884-891, 2014.
Keen et al., International journal of pharmaceutics, 450(1-2): p. 185-196, 2013.
Ku and Dulin, Pharmaceutical development and technology, 17(3): p. 285-302, 2012.
LaFountaine et al., AAPS PharmSciTech, 17(1): p. 43-55, 2016a.
LaFountaine et al., European Journal of Pharmaceutics and Biopharmaceutics, 101: p. 72-81, 2016a.
Leeson, Advanced drug delivery reviews, 101: p. 22-33, 2016.
Loftsson and Brewster, Journal of pharmacy and pharmacology, 62(11): p. 1607-1621, 2010.
Ma et al., Pharmaceutics. 2020.
Marsac et al., Pharmaceutical research, 23(10): p. 2417, 2006.
Meena et al., Journal of Excipients and Food Chemicals, 5(1): p. 1002, 2016.
Meng et al., European Journal of Pharmaceutical Sciences, 71: p. 12-24, 2015.
Miller and Keen, KinetiSol®-based amorphous solid dispersions, in Amorphous Solid Dispersions. Springer. p. 567-577, 2014.
Miller et al., Formulations of deferasirox and methods of making the same. Google Patents, 2019.
Miller et al., Improved formulations of vemurafenib and methods of making the same. Google Patents, 2017.
Miller et al., Journal of Pharmacy and Pharmacology, 68(5): p. 678-691, 2016a.
Miller et al., Spray-Drying, in Formulating poorly water soluble drugs, R. O. Williams III, A. B. Watts, and D. A. Miller, Editors. Springer 2016b.
Parikh et al., Journal of Excipients and Food Chemicals, 5(1): p. 1003, 2016.
Raman et al., Thermally conductive plastic compositions, extrusion apparatus and methods for making thermally conductive plastics. Google Patents, 2016.
Rauwendaal, Plastics Additives and Compounding, 1: p. 21-23, 1999.0
Repka et al., Drug development and industrial pharmacy, 25(5): p. 625-633, 1999.
Sarode et al., European Journal of Pharmaceutical Sciences, 48(3): p. 371-384, 2013.
Shah et al., Journal of pharmaceutical sciences, 102(3): p. 967-981, 2013.
Shenoy, Preparation of filled polymer systems, in Rheology of Filled Polymer Systems. Springer. p. 175-242, 1999.
Touloukian et al., Thermophysical Properties of Matter—The TPRC Data Series—Vol. 1. Thermal Conductivity-Metallic Elements and Alloys. Vol. 1.; 1595, 1970.
Vasconcelos et al., Drug discovery today, 12(23-24): p. 1068-1075, 2007.
Yang, Thermal Conductivity, in Physical properties of polymers handbook, J. E. Mark, Editor.
Springer, 2007.
Yang, Y., Thermal Conductivity, in Physical properties of polymers handbook, J. E. Mark,
Editor. 2007, Springer.
Zhang et al., European journal of pharmaceutics and biopharmaceutics, 82(3): p. 534-544,
2012.

What is claimed is:

1. A method of preparing a pharmaceutical composition comprising subjecting:
(A) an active agent;
(B) a pharmaceutically acceptable polymer; and
(C) a thermally conductive excipient;
to a high energy mixing process at a first temperature and a set speed to obtain the pharmaceutical composition.

2. The method of claim 1, wherein the pharmaceutical composition comprises an amorphous active agent.

3. The method of either claim 1 or claim 2, wherein the pharmaceutical composition comprises an amorphous solid dispersion.

4. The method according to any one of claims 1-3, wherein the active agent is a poorly soluble drug.

5. The method according to any one of claims 1-4, wherein the active agent is a BCS class 2 drug.

6. The method according to any one of claims 1-4, wherein the active agent is a BCS class 3 drug.

7. The method according to any one of claims 1-4, wherein the active agent is a BCS class 4 drug.

8. The method according to any one of claims 1-7, wherein the active agent is an agent which undergoes degradation at an elevated temperature in a formulation process.

9. The method according to any one of claims 1-8, wherein the active agent is chemically sensitive to temperature.

10. The method according to any one of claims 1-9, wherein the active agent is chemically sensitive to shear.

11. The method according to any one of claims 1-10, wherein the active agent does not undergo degradation in the high energy mixing process when the thermally conductive excipient is added to the pharmaceutical composition.

12. The method according to any one of claims 1-11, wherein the active agent is an agent with a melting point of greater than 200° C.

13. The method according to any one of claims 1-12, wherein the active agent is selected from anticancer agents, antifungal agents, psychiatric agents such as analgesics, consciousness level-altering agents such as anesthetic agents or hypnotics, nonsteroidal anti-inflammatory agents (NSAIDS), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

14. The method according to any one of claims 1-13, wherein the active agent is an anti-inflammatory agent.

15. The method of claim 14, wherein the anti-inflammatory agent is a CCR1 antagonist.

16. The method according to any one of claims 1-13, wherein the active agent is an antiepileptic.

17. The method of claim 16, wherein the antiepileptic is a sodium channel blocker.

18. The method according to any one of claims 1-17, wherein the pharmaceutical composition comprises from about 5% w/w to about 90% w/w of the active agent.

19. The method according to any one of claims 1-18, wherein the pharmaceutical composition comprises from about 10% w/w to about 50% w/w of the active agent.

20. The method according to any one of claims 1-19, wherein the pharmaceutical composition comprises from about 20% w/w to about 40% w/w of the active agent.

21. The method according to any one of claims 1-18, wherein the pharmaceutical composition comprises from about 50% w/w to about 90% w/w of the active agent.

22. The method according to any one of claims 1-18 and 21, wherein the pharmaceutical composition comprises from about 60% w/w to about 80% w/w of the active agent.

23. The method according to any one of claims 1-22, wherein the pharmaceutically acceptable polymer is a cellulosic polymer.

24. The method of claim 23, wherein the cellulosic polymer is a neutral cellulosic polymer.

25. The method of claim 23, wherein the cellulosic polymer is a charged cellulosic polymer.

26. The method of claim 23, wherein the cellulosic polymer is hypromellose acetate succinate

27. The method according to any one of claims 1-22, wherein the pharmaceutically acceptable polymer is a neutral non-cellulosic polymer.

28. The method of claim 27, wherein the neutral non-cellulosic polymer comprises a poly(vinyl acetate), polyvinyl caprolactam, poly(vinylpyrrolidone), poly(ethylene glycol), poly(ethylene oxide), poly(vinyl alcohol), or methacrylate unit.

29. The method according to any one of claims 1-28, wherein the pharmaceutically acceptable polymer comprises a poly(vinyl acetate) or a methacrylate unit.

30. The method according to any one of claims 1-29, wherein the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate.

31. The method according to any one of claims 1-30, wherein the pharmaceutical composition comprises from about 5% w/w to about 90% w/w of the pharmaceutically acceptable polymer.

32. The method according to any one of claims 1-31, wherein the pharmaceutical composition comprises from about 30% w/w to about 80% w/w of the pharmaceutically acceptable polymer.

33. The method according to any one of claims 1-32, wherein the pharmaceutical composition comprises from about 50% w/w to about 70% w/w of the pharmaceutically acceptable polymer.

34. The method according to any one of claims 1-33, wherein the thermally conductive excipient is a material that leads to improved thermal conductivity.

35. The method according to any one of claims 1-34, wherein the thermally conductive excipient is a material with a thermal conductivity of greater than 10 W/mK.

36. The method of claim 35, wherein the thermal conductivity is greater than 100 W/mK.

37. The method of claim 36, wherein the thermal conductivity is greater than 200 W/mK.

38. The method of claim 35, wherein the thermal conductivity is from about 100 to about 400 W/mK.

39. The method according to any one of claims 1-38, wherein the thermally conductive excipient is an inorganic material.

40. The method according to any one of claims 1-39, wherein the thermally conductive excipient is an aluminum material.

41. The method of claim 40, wherein the aluminum material is an aluminum inorganic salt.

42. The method of claim 41, wherein the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide.

43. The method of claim 42, wherein the aluminum inorganic salt is potassium aluminum silicate with a coating of titanium dioxide and/or iron oxide.

44. The method of claim 33, wherein the inorganic material is iron oxide, titanium oxide, or silicates.

45. The method according to any one of claims 1-32, wherein the thermally conductive excipient is an organic material.

46. The method of claim 45, wherein the organic material is a dye.

47. The method of claim 46, wherein the dye is carmine, a phthalocyanine, or a diazo compound.

48. The method according to any one of claims 1-47, wherein the pharmaceutical composition comprises from about 0.01% w/w to about 80% w/w of the thermally conductive excipient.

49. The method according to any one of claims 1-48, wherein the pharmaceutical composition comprises from about 0.1% w/w to about 50% w/w of the thermally conductive excipient.

50. The method according to any one of claims 1-49, wherein the pharmaceutical composition comprises from about 1% w/w to about 30% w/w of the thermally conductive excipient.

51. The method according to any one of claims 1-50, wherein the high energy mixing process does not comprise an external heat input.

52. The method according to any one of claims 1-51, wherein the high energy mixing process is a KinetiSol process.

53. The method according to any one of claims 1-52, wherein the set speed is from about 500 rpm to about 6000 rpm.

54. The method according to any one of claims 1-53, wherein the set speed is from about 1000 rpm to about 5000 rpm.

55. The method according to any one of claims 1-54, wherein the set speed is from about 2000 rpm to about 4000 rpm.

56. The method according to any one of claims 1-55, wherein the high energy mixing process comprises mixing the composition comprising two or more set speeds.

57. The method according to any one of claims 1-56, wherein the high energy mixing process is run for a set amount of time.

58. The method of claim 57, wherein the set amount of time is less than 300 seconds.

59. The method of claim 58, wherein the set amount of time is from about 5 seconds to about 300 seconds.

60. The method of claim 59, wherein the set amount of time is from about 5 seconds to about 60 seconds.

61. The method according to any one of claims 1-60, wherein the high energy mixing process comprises a period of prolonged mixing wherein a second temperature does not change by more than 15° C.

62. The method of claim 61, wherein the second temperature does not change by more than 10° C.

63. The method of claim 62, wherein the second temperature does not change by more than 5° C.

64. The method of claim 63, wherein the second temperature does not change by more than 1° C.

65. The method according to any one of claims 1-64, wherein the high energy mixing process comprises a glass transition inflection region and the region of prolonged mixing.

66. The method of claim 65, wherein the glass transition inflection region and the region of prolonged mixing has a time period between the glass transition inflection region and the region of prolonged mixing of less than 20 seconds.

67. The method of claim 65, wherein the time period is less than 15 seconds.

68. The method of claim 67, wherein the time period is less than 10 seconds.

69. The method according to any one of claims 1-68, wherein the high energy mixing process is run until the composition reaches a specific elevated temperature.

70. The method of claim 69, wherein the specific temperature is reached quicker than a composition without the thermally conductive excipient.

71. The method of claim 70, wherein the temperature is reached 30% faster.

72. The method of claim 71, wherein the temperature is reach 10% faster.

73. The method according to any one of claims 1-72, wherein the method further comprises an excipient.

74. The method according to any one of claims 1-73 further comprising quenching the pharmaceutical composition.

75. The method according to any one of claims 1-74 further comprising milling the pharmaceutical composition.

76. The method according to any one of claims 1-75 further comprising formulating the pharmaceutical composition into a unit dose.

77. The method of claim 76, wherein the unit dose is formulated for oral, pulmonary, nasal, topical, transdermal, or parenteral delivery.

78. The method of claim 77, wherein the unit dose is formulated for oral delivery.

79. The method of claim 78, wherein the oral delivery is formulated as a tablet, capsule, or suspension.

80. The method of claim 77, wherein the unit dose is formulated for topical delivery.

81. The method of claim 80, wherein the topical delivery is an emulsion, ointment, or cream.

82. The method of claim 77, wherein the unit dose is formulated for parenteral delivery.

83. The method of claim 82, wherein the parenteral delivery is a suspension, microemulsion, or depot.

84. A pharmaceutical composition comprising:
(A) an active agent;
(B) a pharmaceutically acceptable polymer; and
(C) a thermally conductive excipient;
wherein the pharmaceutical composition comprises an amorphous active agent and the pharmaceutical composition comprises a homogenous mixture of the active agent, the pharmaceutically acceptable polymer, and the thermally conductive excipient.

85. The pharmaceutical composition of claim 84, wherein the pharmaceutical composition comprises an amorphous solid dispersion.

86. The pharmaceutical composition of either claim 84 or claim 85, wherein the active agent is a poorly soluble drug.

87. The pharmaceutical composition according to any one of claims 84-86, wherein the active agent is a BCS class 2 drug.

88. The pharmaceutical composition according to any one of claims 84-86, wherein the active agent is a BCS class 3 drug.

89. The pharmaceutical composition according to any one of claims 84-86, wherein the active agent is a BCS class 4 drug.

90. The method according to any one of claims 84-89, wherein the active agent is an agent which undergoes degradation at an elevated temperature in a formulation process.

91. The pharmaceutical composition according to any one of claims 84-90, wherein the active agent is chemically sensitive to temperature.

92. The pharmaceutical composition according to any one of claims 84-91, wherein the active agent is chemically sensitive to shear.

93. The pharmaceutical composition according to any one of claims 84-92, wherein the active agent does not under degradation in a high energy mixing process when a thermally conductive excipient is added to the pharmaceutical composition.

94. The pharmaceutical composition according to any one of claims 84-93, wherein the active agent is an agent with a melting point of greater than 200° C.

95. The pharmaceutical composition according to any one of claims 84-94, wherein the active agent is selected from anticancer agents, antifungal agents, psychiatric agents such as analgesics, consciousness level-altering agents such as anesthetic agents or hypnotics, nonsteroidal anti-inflammatory agents (NSAIDS), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

96. The pharmaceutical composition according to any one of claims 84-95, wherein the active agent is an anti-inflammatory agent.

97. The pharmaceutical composition of claim 96, wherein the anti-inflammatory agent is a CCR1 antagonist.

98. The pharmaceutical composition according to any one of claims 84-95, wherein the active agent is an antiepileptic.

99. The pharmaceutical composition of claim 98, wherein the antiepileptic is a sodium channel blocker.

100. The pharmaceutical composition according to any one of claims 84-99, wherein the pharmaceutical composition comprises from about 5% w/w to about 90% w/w of the active agent.

101. The pharmaceutical composition according to any one of claims 84-100, wherein the pharmaceutical composition comprises from about 10% w/w to about 50% w/w of the active agent.

102. The pharmaceutical composition according to any one of claims 84-101, wherein the pharmaceutical composition comprises from about 20% w/w to about 40% w/w of the active agent.

103. The pharmaceutical composition according to any one of claims 84-100, wherein the pharmaceutical composition comprises from about 50% w/w to about 90% w/w of the active agent.

104. The pharmaceutical composition according to any one of claims 84-103, wherein the pharmaceutical composition comprises from about 60% w/w to about 80% w/w of the active agent.

105. The pharmaceutical composition according to any one of claims 84-104, wherein the pharmaceutically acceptable polymer is a cellulosic polymer.

106. The pharmaceutical composition of claim 105, wherein the cellulosic polymer is a neutral cellulosic polymer.

107. The pharmaceutical composition of claim 105, wherein the cellulosic polymer is a charged cellulosic polymer.

108. The pharmaceutical composition of claim 107, wherein the cellulosic polymer is hypromellose acetate succinate.

109. The pharmaceutical composition according to any one of claims 84-104, wherein the pharmaceutically acceptable polymer is a neutral non-cellulosic polymer.

110. The pharmaceutical composition of claim 109, wherein the neutral non-cellulosic polymer comprises a poly(vinyl acetate), polyvinyl caprolactam, poly(vinylpyrrolidone), poly(ethylene glycol), poly(ethylene oxide), poly(vinyl alcohol), or methacrylate unit.

111. The pharmaceutical composition according to any one of claims 84-110, wherein the pharmaceutically acceptable polymer comprises a poly(vinyl acetate) or a methacrylate unit.

112. The pharmaceutical composition according to any one of claims 84-111, wherein the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate.

113. The pharmaceutical composition according to any one of claims 84-112, wherein the pharmaceutical composition comprises from about 5% w/w to about 90% w/w of the pharmaceutically acceptable polymer.

114. The pharmaceutical composition according to any one of claims 84-113, wherein the pharmaceutical composition comprises from about 30% w/w to about 80% w/w of the pharmaceutically acceptable polymer.

115. The pharmaceutical composition according to any one of claims 84-114, wherein the pharmaceutical composition comprises from about 50% w/w to about 70% w/w of the pharmaceutically acceptable polymer.

116. The pharmaceutical composition according to any one of claims 84-115, wherein the thermally conductive excipient is a material that leads to improved thermal conductivity.

117. The pharmaceutical composition according to any one of claims 84-116, wherein the thermally conductive excipient is a material with a thermal conductivity of greater than 10 W/mK.

118. The pharmaceutical composition of claim 117, wherein the thermal conductivity is greater than 100 W/mK.

119. The pharmaceutical composition of claim 118, wherein the thermal conductivity is greater than 200 W/mK.

120. The pharmaceutical composition of claim 117, wherein the thermal conductivity is from about 100 to about 400 W/mK.

121. The pharmaceutical composition according to any one of claims 84-120, wherein the thermally conductive excipient is an inorganic material.

122. The pharmaceutical composition according to any one of claims 84-121, wherein the thermally conductive excipient is an aluminum material.

123. The pharmaceutical composition of claim 122, wherein the aluminum material is an aluminum inorganic salt.

124. The pharmaceutical composition of claim 123, wherein the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide.

125. The pharmaceutical composition of claim 124, wherein the aluminum inorganic salt is potassium aluminum silicate with a coating of titanium dioxide and/or iron oxide.

126. The pharmaceutical composition of claim 125, wherein the inorganic material is iron oxide, titanium oxide, or silicates.

127. The pharmaceutical composition according to any one of claims 84-120, wherein the thermally conductive excipient is an organic material.

128. The pharmaceutical composition of claim 127, wherein the organic material is a dye.

129. The pharmaceutical composition of claim 128, wherein the dye is carmine, a phthalocyanine, or a diazo compound.

130. The pharmaceutical composition according to any one of claims 84-129, wherein the pharmaceutical composition comprises from about 0.01% w/w to about 80% w/w of the thermally conductive excipient.

131. The pharmaceutical composition according to any one of claims 84-130, wherein the pharmaceutical composition comprises from about 0.1% w/w to about 50% w/w of the thermally conductive excipient.

132. The pharmaceutical composition according to any one of claims 84-131, wherein the pharmaceutical composition comprises from about 1% w/w to about 30% w/w of the thermally conductive excipient.

133. The pharmaceutical composition according to any one of claims 84-132, wherein the pharmaceutical composition has been processed through a high energy mixing process.

134. The pharmaceutical composition according to any one of claims 84-133 further comprising an excipient.

135. The pharmaceutical composition according to any one of claims 84-134 further comprising milling the pharmaceutical composition.

136. The pharmaceutical composition according to any one of claims 84-135 further comprising formulating the pharmaceutical composition into a unit dose.

137. The pharmaceutical composition according to any one of claims 84-136, wherein the unit dose is formulated for oral, pulmonary, nasal, topical, transdermal, or parenteral delivery.

138. The pharmaceutical composition according to any one of claims 84-137, wherein the unit dose is formulated for oral delivery.

139. The pharmaceutical composition of claim 138, wherein the oral delivery is formulated as a tablet, capsule, or suspension.

140. The pharmaceutical composition according to any one of claims 84-137, wherein the unit dose is formulated for topical delivery.

141. The pharmaceutical composition of claim 140, wherein the topical delivery is an emulsion, ointment, or cream.

142. The pharmaceutical composition according to any one of claims 84-137, wherein the unit dose is formulated for parenteral delivery.

143. The pharmaceutical composition of claim 142, wherein the parenteral delivery is a suspension, microemulsion, or depot.

144. The pharmaceutical composition according to any one of claims 84-143 comprising:
(A) about 3% w/w of the thermally conductive excipient, wherein the thermally conductive excipient is potassium aluminum silicate with a coating of titanium dioxide and/or iron oxide;
(B) about 33% w/w of the active agent, wherein the active agent is CCR1 antagonist, and
(C) about 64% w/w of the pharmaceutically acceptable polymer, wherein the pharmaceutically acceptable polymer is hypromellose acetate succinate.

145. A pharmaceutical composition prepared according to the methods described in any one of claims 1-83.

146. A method of treating a disease or disorder comprising administering to the patient a therapeutically effective amount of a pharmaceutical composition according to any one of claims 84-144 wherein the active agent is effective to treat the disease or disorder.

147. A pharmaceutical composition comprising:
(A) a pharmaceutically acceptable polymer; and
(B) a thermally conductive excipient;
wherein the pharmaceutical composition comprises a homogenous mixture of the pharmaceutically acceptable polymer and the thermally conductive excipient.

148. A method of preparing a composition comprising:
(A) a pharmaceutically acceptable polymer; and
(B) a thermally conductive excipient;
to a high energy mixing process at a first temperature and a set speed to obtain the composition.
